SEQUENCE LISTING (1 sequence)

SEQ ID NO: 1 - DNA, 1500 bp, Artificial Sequence
  Description of Artificial Sequence/note = synthetic construct
atgggtgcgagagcgtcagtattaagcgggggaaaattagatgcatgggaaaaaattcggttaaggccagggggaaagaaaaaatatagactaaaacatctagtatgggcaagcagggagctggaaagatttgcacttaaccctggccttttagaaacatcagaaggctgtaaacaaataatgggacagctacaaccagctcttcagacaggatcagaagaacttagatcattatataatacagtagcaaccctctattgtgtacatcaaaggatagaggtaaaagacaccaaggaagctttagagaagatagaggaagaacaaaacaaaagtaagcaaaagacacagcaggcagcagctgacacaggaaacagcagccaggtcagccaaaattaccctatagtgcagaatctacaagggcaaatggtacaccaggccatatcacctagaactttgaatgcatgggtaaaagtaatagaagaaaaggctttcagcccagaagtaatacccatgttttcagcattatcagaaggagccaccccacaagatttaaacaccatgctaaacacagtggggggacatcaagcagccatgcaaatgttaaaagataccatcaatgaggaagctgcagaatgggataggttacatccagtacatgcagggcctattccaccaggccagatgagagaaccaaggggaagtgacatagcaggaactactagtacccttcaggaacaaataggatggatgacaagcaacccacctatcccagtgggagaaatctataaaagatggataatcctgggattaaataaaatagtaagaatgtatagccctgtcagcattttggacataagacaagggccaaaagaaccctttagagactatgtagacaggttctttaaaactctaagagctgagcaagctacacaggatgtaaaaaattggatgacagaaaccttgttggtccaaaatgcgaacccagattgtaagaccattttaaaagcattaggaccaggggctacactagaagaaatgatgacagcatgtcagggagtgggaggacccagccataaagcaagagttttggctgaggcaatgagccaagcaacaaatgcagccataatgatgcagagaggcaattttaagggccaaagaagaattattaagtgtttcaactgtggcaaagaaggacacatagccagaaattgcagggcccctaggaaaaagggctgttggaaatgtggaaaggaaggacaccaaatgaaagactgcactgaaagacaggctaattttttagggaaaatttggccttccaacaaggggaggccagggaattttcttcagagcagaccagagccaacagccccaccagcagagagcttcgggttcggggaggagataaccccctctccgaagcaggagcagaaagacaaggaactgtatcctcctttagcttccctcaaatcactctttggcaacgaccccttgtcacaataa

(19) United States
(12) Patent Application Publication
(10) Pub. No.: US 2008/0050718 A1
(43) Pub. Date: Feb. 28, 2008

(54) Methods, Articles, and Compositions for Identifying Oligonucleotides

(76) Inventors: Raymond F. Gesteland, Salt Lake City, UT (US); John F. Atkins, Salt Lake City, UT (US); Olga V. Matveeva, Salt Lake City, UT (US); Svetlana A. Shabalina, Bethesda, MD (US)

Correspondence Address: NEEDLE & ROSENBERG, P.C., SUITE 1000, 999 PEACHTREE STREET, ATLANTA, GA, 30309-3915, US

(21) Appl. No.: 10/583,198

(22) PCT Filed: Nov. 15, 2004
(86) PCT No.: PCT/US04/38092

§ 371 Date: May 31, 2007

Related U.S. Application Data
(60) Provisional application No. 60/519,911, filed on Nov. 14, 2003.

(51) Int. Cl. C12Q 1/68; C07H 21/04; C12Q 1/70

(52) U.S. Cl. 435/5; 435/6; 536/24.33

(57) ABSTRACT

There are many situations where oligonucleotides that efficiently bind a target DNA or RNA are desired. These oligonucleotides can be used for a variety of purposes, including antisense, diagnostics, and array generation. While researchers have worked for many years to identify algorithms and methods for predicting the oligonucleotides that will bind the target with the highest efficiency, better prediction methods are needed. Disclosed are methods, articles, machines, and compositions that aid in identifying oligonucleotides and sets of oligonucleotides that will efficiently bind a target nucleic acid molecule. Also disclosed are optimized sets of oligonucleotides that bind HIV-1 genomic RNA or DNA, such as the GAG RNA, and methods of using them.

### I. ACKNOWLEDGEMENTS

1. This invention was made with government support under federal grant RO1-GM61200 and GM48152 awarded by the NIH. The Government has certain rights to this invention.

### II. BACKGROUND

2. There are many situations where oligonucleotides that efficiently bind a target DNA or RNA are desired. These oligonucleotides can be used for a variety of purposes, including antisense, diagnostics, and array generation. While researchers have worked for many years to identify algorithms and methods for predicting the oligonucleotides that will bind the target with the highest efficiency, better prediction methods are needed. Disclosed are methods, articles, machines, and compositions that aid in identifying oligonucleotides and sets of oligonucleotides that will efficiently bind a target nucleic acid molecule. Also disclosed are optimized sets of oligonucleotides that bind HIV-l genomic RNA or DNA,, such as the GAG RNA, and methods of using them.

### III. SUMMARY

3. Disclosed are methods and compositions related to methods, compositions, and articles related to identification of oligonucleotides designed to hybridize with a target nucleic acid.

### IV. BRIEF DESCRIPTION OF THE DRAWINGS

4. The accompanying drawings, which are incorporated in and constitute a part of this specification, illustrate several embodiments and together with the description illustrate the disclosed compositions and methods.

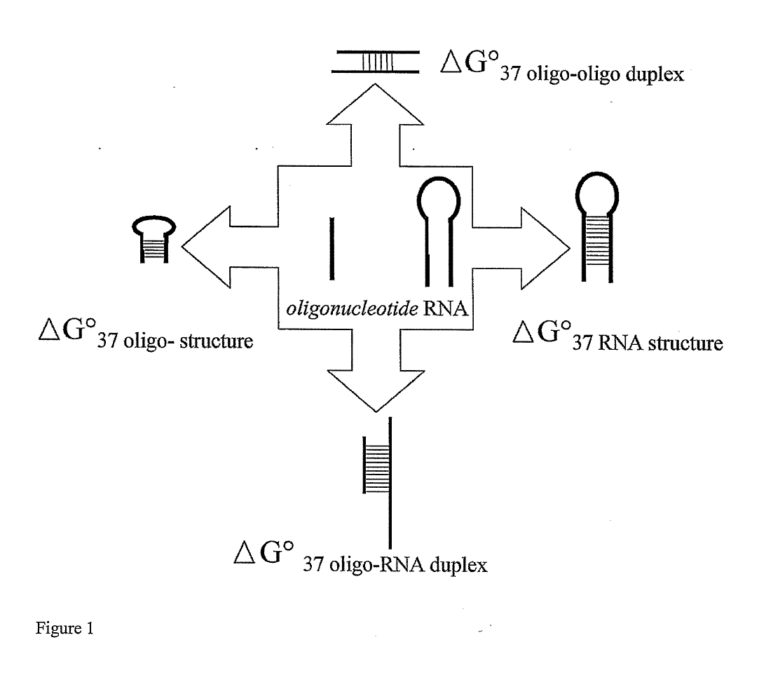

5. FIG. 1 shows a scheme of oligonucleotide-target RNA interaction, which shows thermodynamic factors that can influence oligonucleotide RNA hybridization intensity.

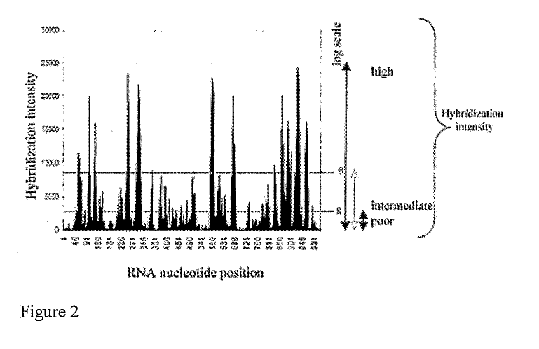

6. FIG. 2 shows an RNA hybridization intensity profile for the set of oligonucleotides (20 mers) that was used for creation of the first dataset. The hybridization intensity is shown for each oligonucleotide in relation to its position in the target RNA. For statistical analysis, the oligonucleotides were categorized into groups according to hybridization intensity. The small arrow represents the group with low hybridization intensity; medium sized arrow, intermediate; and large arrow with high.

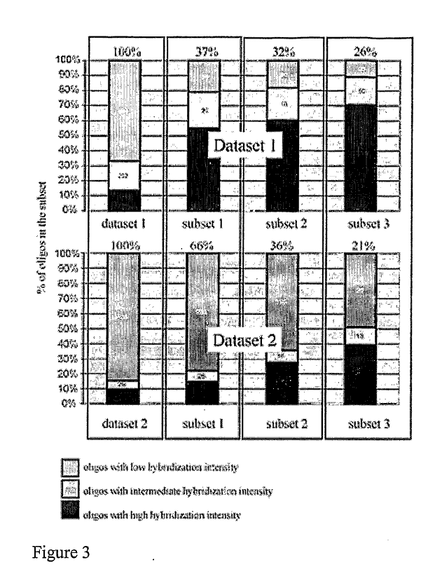

7. FIG. 3 shows a relationship between calculated thermodynamic parameters and hybridization intensity of the oligonucleotides with their target RNA.

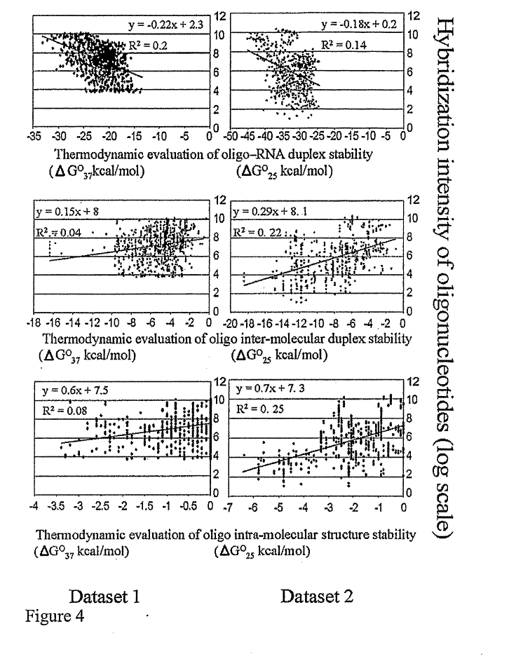

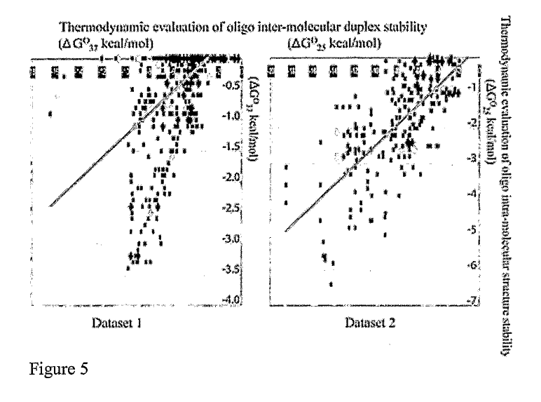

8. FIG. 4 shows a categorization of oligonucleotides into subsets according to their thermodynamic properties. The percentage of oligonucleotides with RNA hybridization intensity higher than the defined threshold in each subset is shown. The code is the same as in FIG. 2. Numbers of oligonucleotides in each subgroup are printed on highlighted parts of the columns. The proportion of oligonucleotides in each subset versus the total number of oligonucleotides in the relevant dataset is shown above each column. Subset 1 contains oligo-probes that can form stable duplexes with RNA dG°₂₅≦29 kcal/mol; subset 2 contains the oligo-probes that can form stable duplexes with RNA dG°₂₅≦29 kcal/mol with unstable intermolecular oligo self-structures dG°₂₅≧8 kcal/mol; and subset 3 contains oligo-probes that can form stable duplexes with RNA dG°₂₅≦29 kcal/mol but which form both unstable inter- and intra-molecular self-structures (dG°₂₅≧8 kcal/mol for inter-molecular structures and dG°₂₅≧1.1 kcal/mol for intra-molecular structures).

9. FIG. 5 shows a relationship between thermodynamic evaluations of oligonucleotide inter- and intra-molecular pairing potentials (x andy axes, respectively). Medoum gray squares represent the group with low hybridization intensity; light gray, intermediate; and dark grey with high.

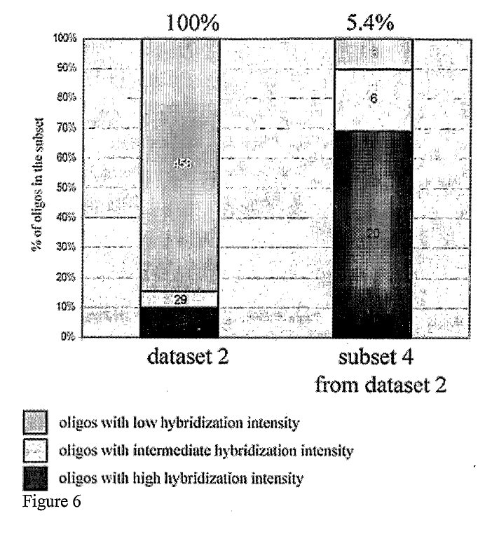

10. FIG. 6 shows a categorization of oligonucleotides into subsets according to their thermodynamic properties. Two sets of oligonucleotides in dataset 2 are shown. The first set represents all oligonucleotides in the dataset, while the second represents only the fraction with certain thermodynamic properties. The proportion of oligonucleotides in each subset versus the total number of oligonucleotides in dataset 2 is shown above each column. The percentage of oligonucleotides with RNA hybridization intensity higher than the defined threshold in each set is also shown. The code is the same as in FIG. 2. Numbers of oligonucleotides in each subgroup are printed on highlighted parts of the columns. Subset 4 contains oligo-probes that can form stable duplexes with RNA dG°₂₅≦35 kcal/mol but which form both unstable inter- and intra-molecular self-structures (dG°₂₅≧8 kcal/mol for inter-molecular structures and dG°₂₅≧1.1 kcal/mol for intra-molecular structures).

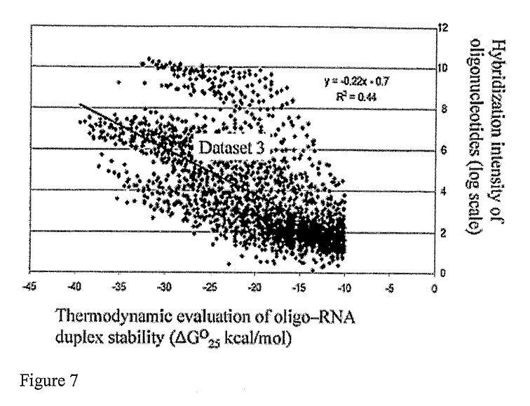

11. FIG. 7 shows a relationship between calculated values of dG°₂₅ of DNA-RNA duplex stability and hybridization intensities of the oligonucleotides with their target RNA for the subset of oligo-probes with little self-structure from dataset 3.

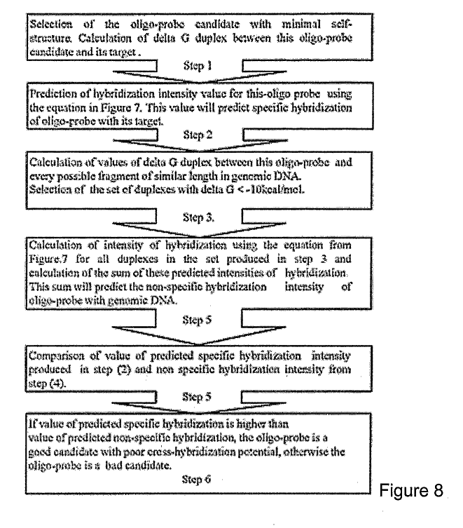

12. FIG. 8 shows a scheme for evaluation of cross-hybridization potentials of oligo-probe candidates.

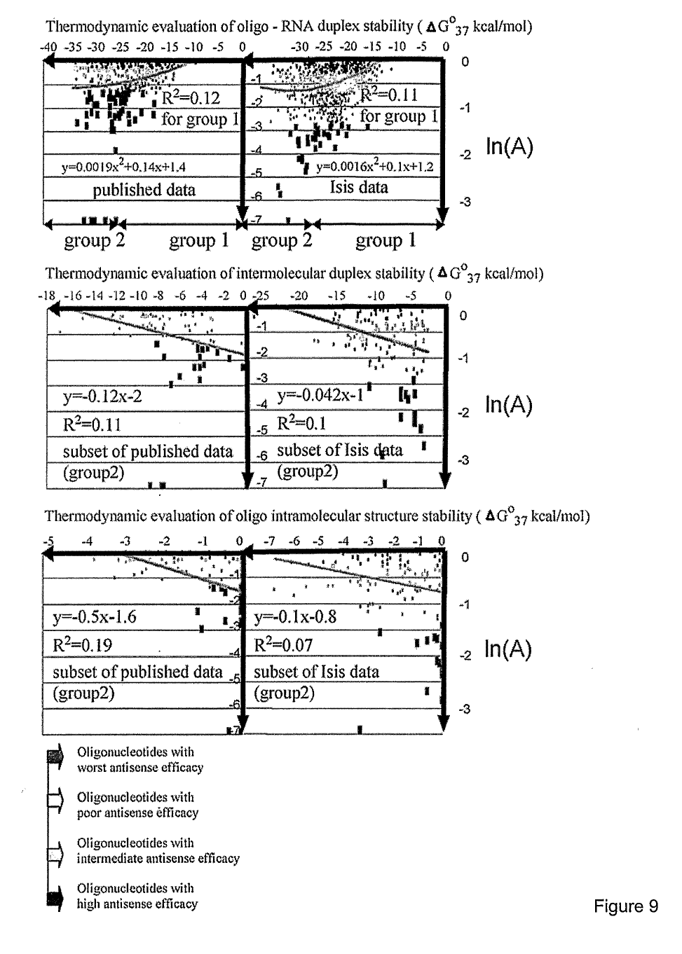

13. FIG. 9 shows scatter plots showing the relationship between thermodynamic parameters and antisense oligonucleotide activities from both databases. Activity values (A) are expressed as the ratio of the level of a particular mRNA or protein measured in cells treated with an antisense oligonucleotide, to the level of the same mRNA or protein in untreated cells. Linear or non-linear trend lines are shown in each scatter plot.

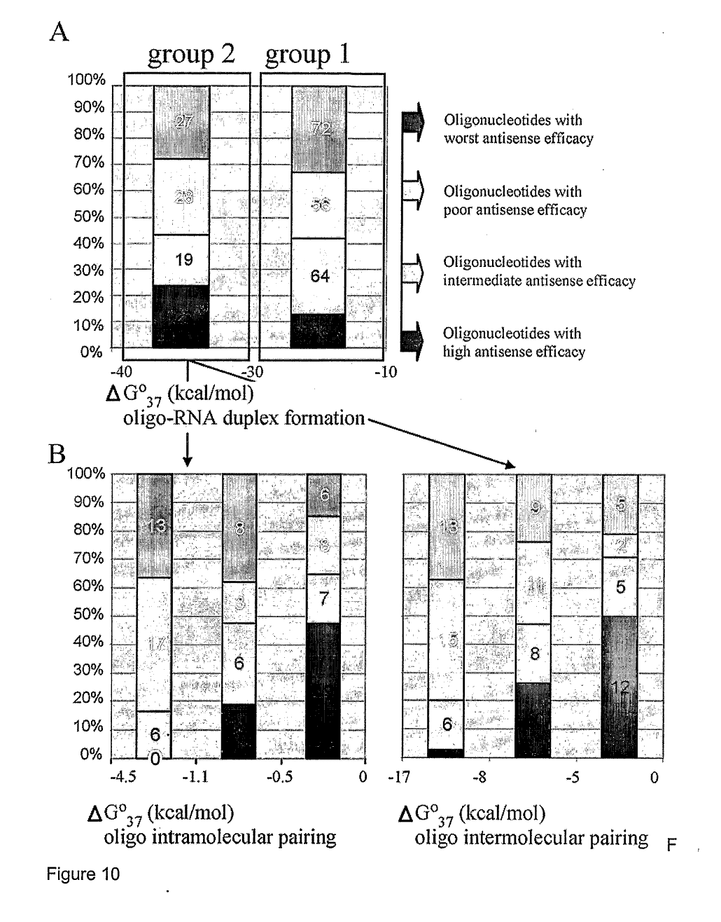

14. FIG. 10 shows a relationship between thermodynamic parameters and antisense oligonucleotide activities determined for the web database. (A) Oligo nucleotides were categorized into two groups according to calculated values of dG°₃₇ for DNA-RNA duplex formation. Group 1 contains oligonucleotides that form more stable duplexes, and group 2 contains oligonucleotides that form less stable duplexes with target RNA. (B) Group 1 oligonucleotides separated on the basis of the calculated dG°₃₇ for oligonucleotide intra-molecular pairing. (C) Group 1 oligonucleotides separated on the basis of the calculated dG°₃₇ for oligonucleotide inter-molecular pairing. The numbers of oligonucleotides in each subgroup are indicated in the relevant highlighted segments.

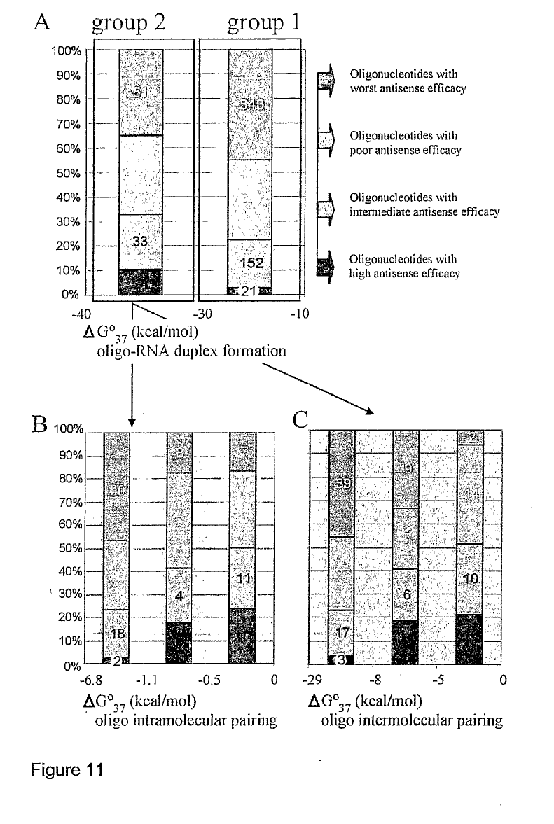

15. FIG. 11 shows a relationship between thermodynamic parameters and antisense oligonucleotide activities determined for the Isis database. Oligonucleotides were categorized into two groups according to the calculated value of dG°₃₇ of duplex formation. (A) Group 1 contains oligonucleotides that form more stable duplexes and group 2 contains oligonucleotides that form less stable duplexes with target RNA. (B) Group 1 oligonucleotides were further separated based on the calculated dG°₃₇ for oligonucleotide intra-molecular pairing. (C) Group 1 oligonucleotides were further separated based on the calculated dG°₃₇ for oligonucleotide inter-molecular pairing. For each set, oligonucleotides were separated into subgroups according to their antisense efficacy. The numbers of oligonucleotides in each subgroup are on the relevant highlighted segments.

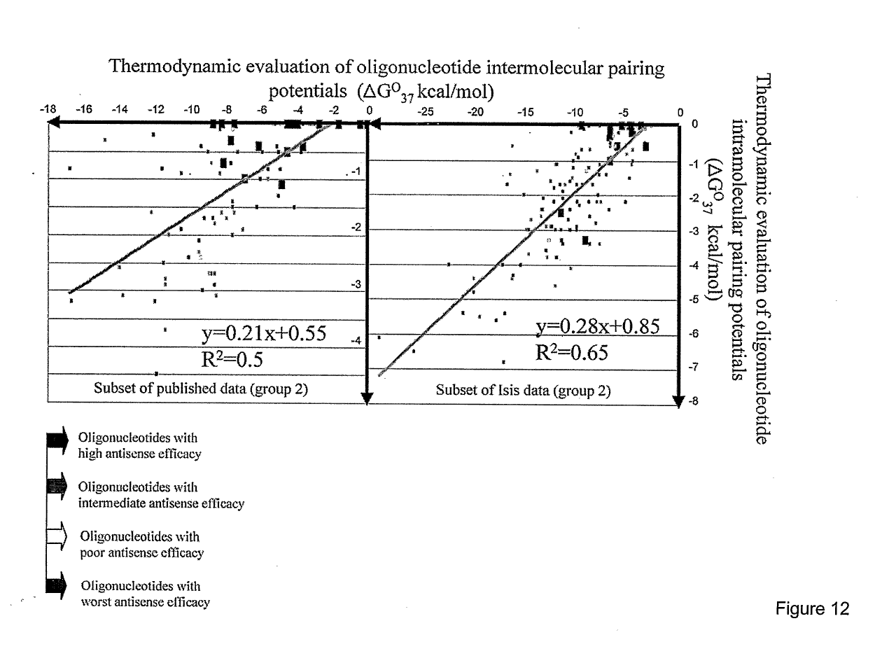

16. FIG. 12 shows a relationship between thermodynamic evaluations of oligonucleotide inter- and intra-molecular pairing potentials (x- and y-axis, respectively). The trend line is shown in each scatter plot.

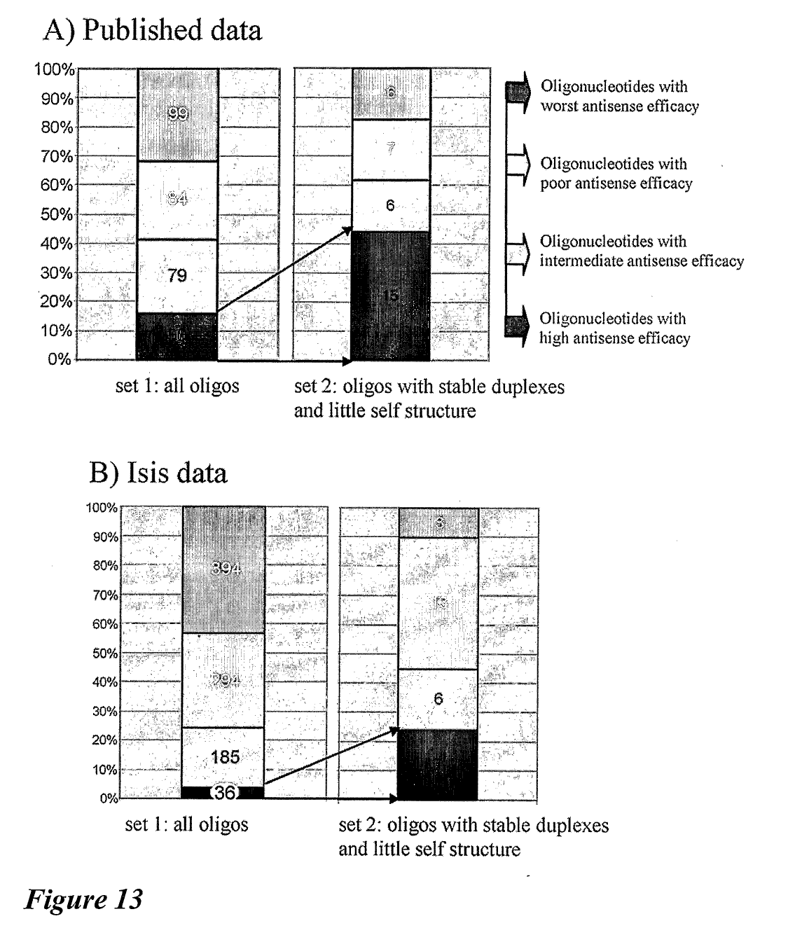

17. FIG. 13 shows a relationship between thermodynamic parameters and antisense oligonucleotide activities from both databases. (A) Data from the published antisense oligonucleotide experiments. (B) Unpublished data from Isis Pharmaceuticals. The numbers of oligonucleotides in each subgroup are on the relevant segments. Set 1 contains all oligonucleotides in each database. Set 2 includes only oligonucleotides predicted to form very stable duplexes (dG°₃₇≦30 kcal/mol) and those with the least possibility for self-structure (dG°₃₇≧5 kcal/mol for inter-molecular oligonucleotide pairing and dG°₃₇≧1 kcal/mol for intra-molecular pairing).

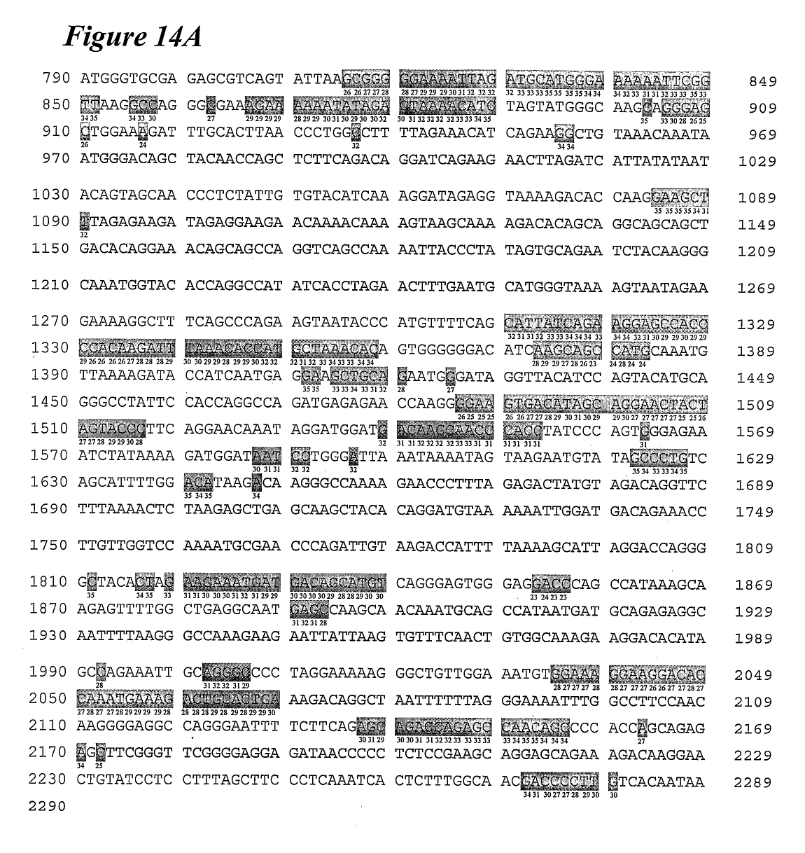

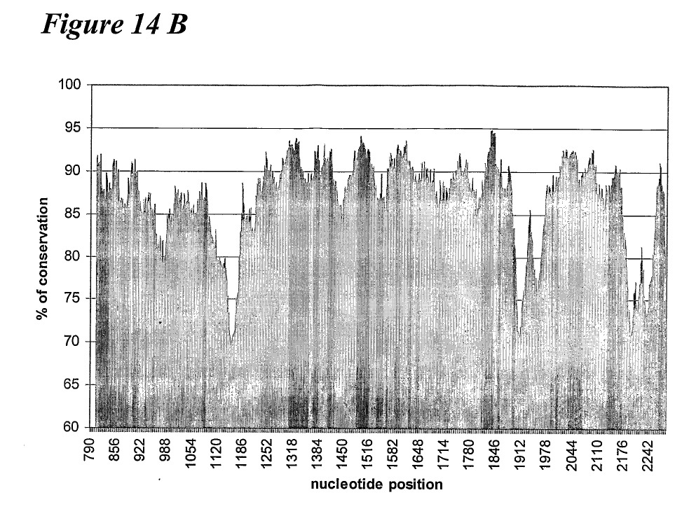

18. FIG. 14 shows a consensus GAG sequence and a plot of conservation with a 30 nucleotide window. FIG. 14A shows Gag consensus sequence. Last nucleotides in the theoretically optimal target regions are highlighted. The range of fragments that were analyzed was from 23 to 35-mers. The length of optimal region is shown below the highlighted nucleotide. Only numbers for shortest regions in the sets that correspond to each highlighted nucleotide are shown. FIG. 14B shows a Gag plot of conservation made with window of 30 nucleotides and step 1. Average conservation for each consequent 30 nucleotides is shown. Conserved regions that are thermodynamically optimal for oligonucleotide targeting are highlighted.

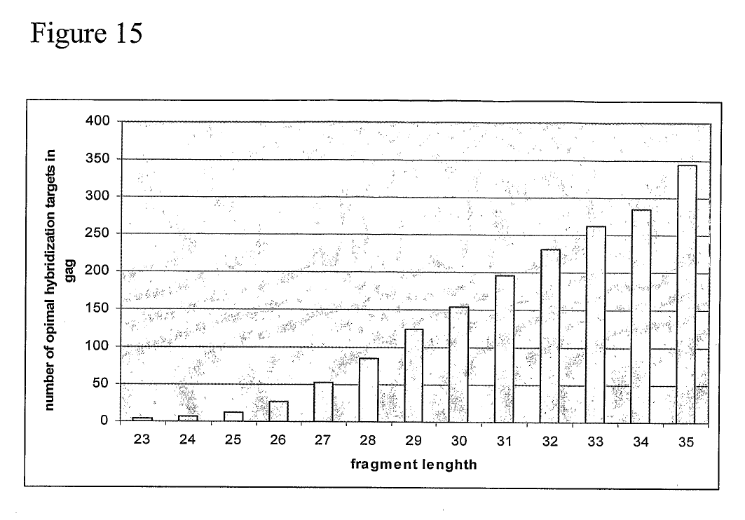

19. FIG. 15 shows the number of theoretically optimal RNA targets obtained with each possible length of oligonucleotide, in the range from 23 to 35-mers.

### V. DETAILED DESCRIPTION

20. Disclosed are methods, compositions, and articles that allow for the efficient identification of oligonucleotides that will hybridize better with target sequences. These methods, compositions, and articles are based on the disclosed understanding of certain thermodynamic parameters and how they relate to each other and how they affect the efficient binding of a given oligo for a target nucleic acid. One nucleic acid binds or hybridizes with another nucleic acid based on the ability of the two nucleic acids to form base pairs with each producing a duplex or double stranded DNA molecule. Whether two nucleic acids hybridize is a combination of the thermodynamic properties of four separate interactions that take place or can take place between the first nucleic acid or oligo, for example, and the second nucleic acid, or target. These four parameters are shown in FIG. 1. The first parameter is the Gibbs free energy, delta G, or dG of the interaction between the oligo and the target RNA molecule. This is the dG of the desired interaction, or the sub part of the total energy that arises when the oligo and the target come together that is due to the actual interactions between the oligo and the target. This parameter can be represented as dG°_{oligo-RNA duplex}. Another parameter that can effect the overall dG of the target and oligo coming together is the self structure of the oligo itself, the ability of the oligo to form secondary and tertiary structures, such as hairpins or pseudoknots. This parameter can be represented as dG°_{oligo-structure}. A third parameter that can effect the overall dG for the oligo-target interaction is the dG of the oligo forming dimers or multimers with itself. This third parameter can be represented as dG°_{oligo-oligo dimer}. Lastly, the fourth parameter that can effect the overall dG of oligo and target is the self structure of the target RNA molecule itself. This fourth parameter can be represented as dG°_{RNA structure}. It is understood that the dG°_{oligo-RNA duplex} can be considered a promotion force behind the overall force bring the oligo and the target together and that the dG°_{oligo-structure}, dG°_{oligo-oligo dimer}, and dG°_{RNA} structure can be considered negative forces, in essence reducing the ability of the oligo and target to come together. These parameters are in essence competing energies for the energy of duplex formation. Oligo intra- or inter-molecular structure can compete with oligo-target duplex formation and result in low hybridization intensity. Extensive secondary structure of the target can also limit this efficiency. Disclosed herein it is shown that thermodynamic considerations of the relative stability of oligo-target duplexes and both oligo intra- and inter-molecular self-structures, without consideration of target secondary structure, can be sufficient for selection of oligo-probes that are efficient target binders. In other embodiments the structure of the target nucleic acid can also be considered. The disclosed methods, articles, and compositions, are provide guidelines for how to weight each of these parameters and how to analyze a given oligo's likelihood of being an oligo having a relatively strong overall affinity for a target nucleic acid molecule, such as an RNA molecule. Disclosed are methods that allow for the identification of sets of oligos that will have a higher probability of having a better overall affinity for binding the target nucleic acid. Also disclosed are compositions and articles, as well as machines that can be used in the disclosed methods. In certain embodiments, general methods that allow for the identification of any oligo for a specific target region are disclosed. In addition, methods that allow for the identification optimal oligos for a target even when the target has varying regions are disclosed.

22. In certain embodiments the disclosed methods are designed for identifying oligos that bind at set temperatures, such as 37° C. or 25° C. Furthermore, in certain methods, the design is for conditions where there is higher ionic strength, for example, higher than the ionic strength of a typical PCR reaction and at relatively low temperatures, for example, under about 65° C. This is because existing methods that predict effective oligonucleotide primers for identifying primers for these other conditions, such as picking primers for PCR reactions for a particular DNA template, work well for those applications because the primers will be employed under relatively stringent conditions. Thus PCR experimental primer design greatly simplifies the prediction problem: hybridization is performed at relatively low ionic strength and high temperature. Under these relatively stringent conditions, oligonucleotide and target secondary structures and oligo-oilgo duplex/multimer formation (dG°_{oligo-structured}dG°_{RNA structure}, and dG°_{oligo-oligo dimer} are relatively unimportant. However, as discussed herein these structures become much more important at temperatures closer to and around 37° C. These lower temperatures of oligo-RNA hybridization are frequently used in a number of different RNA detection assays and so efficient prediction of preferred oligo sets are desired. The disclosed methods, compositions, and articles, are designed to increase the efficiency of oligonucleotide design for target hybridization at around 37° C. Methods for identifying the optimal parameters for a given temperature are known and can be found in U.S. patent application Ser. No. 10/374,253, filed on Feb. 26, 2003, for “Methods for designing oligo-probes with high hybridization efficiency and high antisense activity” by Olga Matveeva, and which is herein incorporated by reference in its entirety and at least for material related to methods for determining the threshold levels for the thermodynamic parameters at any given temperature and for material related to the identification and use of these parameters.

23. Thus, optimization of probe design for array-based experiments requires improved predictability of oligonucleotide hybridization behavior. Currently, designing oligonucleotides capable of interacting efficiently and specifically with the relevant target is not a routine procedure. Multiple examples demonstrate that oligonucleotides targeting different regions of the same RNA differ in their hybridization ability. Disclosed are thermodynamic evaluations of oligo-target duplex or oligo self-structure stabilities and their effect on probe design. Statistical analysis of large sets of hybridization data reveals that certain thermodynamic evaluation parameters of oligonucleotide properties can be used to avoid poor RNA or target binders. Thermodynamic criteria for the selection of 20 and 21 mers, which, with high probability, interact efficiently and specifically with their targets, are disclosed herein, and used as an example, but it is understood that the disclosed methods can be used for primers of any length. For example, the design of longer oligonucleotides can also be facilitated by the same calculations of dG°T values for oligo-target duplex or oligo self-structure stabilities and similar selection schemes.

24. Many techniques of molecular biology require interaction of oligonucleotides with DNA or RNA as a basic step. Oligonucleotide array gene expression monitoring or antisense-mediated gene down-regulation are examples. Poor interaction of an oligonucleotide with its target can significantly affect the efficiency of these processes.

25. The disclosed methods were identified and confirmed by utilizing, comparing, and synthesizing data generated from two existing but different ways for monitoring hybridization efficiency for a given oligo-target interaction. One is the brute force method, capable today because of array technology, of individually testing the binding of each oligo to the target sequence and comparing it to the binding of each other oligo to the target sequence. The second way is to use programs to predict the binding efficiency of a given oligo for a target nucleic acid. When each of these methods is employed for a given oligo or set of oligos and a given target, different sets of oligos are identified. The disclosed methods are based on the detailed and intricate comparison of multiple iterations of both types of data for a given oligo set and given target sequence. This allowed for the disclosed constraints or weighting coefficients, that can be placed on the various parameters discussed herein that allow for the increased success of predicting efficient oligonucleotide binders, using existing methods for determining their thermodynamic parameters.

26. Oligonucleotide scanning arrays permit monitoring of the efficiency of hybridization simultaneously for many, or all, target regions of a particular RNA. RNA target affinity can also be measured for oligonucleotides of different length and self-structure in one hybridization experiment (Williams, J. C., et al., (1994), Nucleic Acids Res., 22, 1365-1367; Southern, E. M., et al., (1994), Nucleic Acids Res., 22, 1368-1373; Southern, E. M. (2002), Methods Mol. Biol., 170, 1-15; Sohail, M., et al., (1999), RNA, 5, 646-655; Sohail, M. and Southern, E. M. (2002), Methods Mol. Biol., 170, 181-199; Sohail, M., et al., (2002), Nucleic Acids Res., 29, 2041-2051; Southern, E., Mir, K. and Shchepinov, M. (1999), Nature Genet., 21, 5-9), so these arrays can be very useful for the statistical study of oligonucleotide-related factors that influence an oligonucleotide's ability to hybridize with target RNA or DNA.

27. Software for the calculation of the thermodynamic factors that are important for the prediction of oligonucleotide hybridization behavior was created some time ago (Mathews, D. H., et al., (1999), RNA, 5, 1458-1469). The program OligoWalk calculates thermodynamic factors related to stabilities of oligonucleotide-target duplex, oligonucleotide intra- or inter-molecular self-structures and target RNA or DNA secondary structure.

28. The disclosed methods can be used to identify preferred antisense molecules for desired targets. Antisense oligonucleotides are used for therapeutic applications and in functional genomic studies. In practice, however, many of the oligonucleotides complementary to an mRNA have little or no antisense activity. Theoretical strategies to improve the ‘hit rate’ in antisense screens will reduce the cost of discovery and may lead to identification of antisense oligonucleotides with increased potency. Statistical analysis performed on data collected from more than 1000 experiments with phosphorothioate-modified oligonucleotides revealed that the oligo-probes, which form stable duplexes with RNA (dG°₃₇≦about−30 kcal/mol) and have small self-interaction potential, are more frequently efficient than molecules that form less stable oligonucleotide-RNA hybrids or more stable self-structures. To achieve optimal statistical preference, the values for self-interaction should be (dG°₃₇≧about−8 kcal/mol for inter-oligonucleotide pairing and (dG°₃₇)≧about−1.1 kcal/mol for intra-molecular pairing are disclosed. Selection of oligonucleotides with these thermodynamic values in disclosed traditional calculated hybridization oligonucleotides would have increased the ‘hit rate’ by as much as 6-fold.

29. Antisense oligonucleotides in current use are typically modified DNA molecules that hybridize to complementary MRNA and inhibit expression of its encoded product. In principle, the antisense approach is universal and specific. It can be used to inhibit expression of any mRNA, and a single protein isoform can be shut down without affecting closely related proteins. Antisense oligonucleotides are used for therapeutic applications and in functional genomic studies. In practice, however, many of the oligonucleotides complementary to an mRNA have little or no antisense activity. Typically, several oligonucleotides are synthesized and tested and only some are active. Theoretical strategies to improve the ‘hit rate’ in antisense screens will reduce the cost of discovery and may lead to identification of antisense oligonucleotides with increased activity or potency. Theoretical prediction of RNA target sites for active oligonucleotides is related to the development of algorithms that can locate single-stranded regions in RNA secondary structure models (Sczakiel, G. and Tabler, M. (1997), Methods Mol. Biol., 74, 11-15; Patzel, V., et al., (1999), Nucleic Acids Res., 27, 4328-4334; Lehmann, M. J., et al., (2000), Nucleic Acids Res., 28, 2597-2604; Scherr, M., et al., (2000), Nucleic Acids Res., 28, 2455-2461; Sczakiel, G. (2000), Front. Biosci., 5, 194-201; Ding, Y. and Lawrence, C. E. (2002), Nucleic Acids Res., 29, 1034-1046; Mathews, D. H., et al., (1999), RNA, 5, 1458-1469, of which are incorporated herein, at least for material related to nucleic acid structure). There is some experimental evidence that oligonucleotides designed to target these non-structured RNA regions are indeed frequently efficient in down regulation of particular gene products (Sczakiel, G. and Tabler, M. (1997), Methods Mol. Biol, 74, 11-15; Patzel, V., et al., (1999), Nucleic Acids Res., 27, 4328-4334; Lehmann, M. J., et al., (2000), Nucleic Acids Res., 28, 2597-2604; Scherr, M., et al., (2000), Nucleic Acids Res., 28, 2455-2461; Sczakiel, G. (2000), Front. Biosci., 5, 194-201). It is not known how much oligonucleotide self-pairing decreases the ‘hit-rate’. Software for calculation of thermodynamic properties of oligonucleotide structure, target RNA structure and duplex formation has been developed (Mathews, D. H., et al., (1999), RNA, 5, 1458-1469). Thus, disclosed are methods and articles as well as compositions that address these problems.

A. Methods
1. General Method for a Target Sequence

30. Limited work has been performed on simultaneous combinations of thermodynamic and homology analyses for predicting optimal universal targets in related RNA sequences for oligonucleotide hybridization (Lucas, K., et al., (1991) Comput Appl Biosci, 7, 525-529; Dopazo, J., et al., (1993) Comput Appl Biosci, 9, 123-125; Proutski, V. and Holmes, E. C. (1996) Comput Appl Biosci, 12,253-255; Kel, A., et al., (1998) Bioinformatics, 14,259-270; and Gibbs, A., et al., (1998) J Virol Methods, 74, 67-76). In the disclosed scheme are experimentally derived thermodynamic discriminatory steps. Decisions about the suitability of a particular target region are determined by a set of thresholds, which were found after analysis of the efficiency of oligonucleotides in the experimental databases Matveeva, O. V., et al. (2003) Nucleic Acids Res, 31, 4211-4217, Matveeva, O. V., et al (2003). Nucleic Acids Res, 31, 4989-4994. Several experimental databases were analyzed: databases of hybridization performed with large sets of arrayed oligonucleotides that contain data for every overlapping 20 or 21 nt probe to target RNA sequence and databases of antisense experiments. The latter databases contain information of the levels of down-regulation of particular gene products in cells after treatment with antisense oligonucleotides. Statistical analysis of data collected from more than 1000 experiments with antisense DNA oligonucleotides, revealed that the chance of an oligonucleotide being efficient in shutting down a specific gene is greater for molecules that have high RNA pairing potential and low self-interaction potential. Oligonucleotides that form stable duplexes with RNA (free energies (ΔG°₃₇)≦30 kcal/mol) and little self structure are statistically more likely to be active than molecules, which form less stable oligonucleotide-RNA hybrids or more stable self-structures. For the achieving of optimal statistical preference the values for self-interaction should be (ΔG°₃₇)≧−8 kcal/mol for inter- oligonucleotide pairing and (ΔG°₃₇)≧−1.1 kcal/mol for intra-molecular pairing. Selection of oligonucleotides with these thermodynamic values in the analyzed experiments would have increased the proportion of active oligonucleotides by as much as six folds. Since efficient binding of antisense oligonucleotide with target mRNA is a pre-requisite for RNase H mediated inactivation of gene expression, the same set of thermodynamic thresholds can be applied for selecting promising oligonucleotides for hybridization probes when similar conditions are used.

31. Thus, in certain embodiments the methods involve a filtering step or steps which increases the likelihood that any given oligonucleotide within the identified set will be a relatively efficient binder of the target. The following general steps of the methods follow.

32. A target nucleic acid is identified and the size of the desired oligos is identified, such as 20, or 21, or 30. It is understood that these identifications may form part of the overall method, but they do not have to be performed as part of the method, for example, these identifications could have taken place previously, in another context. However, one starts with a target nucleic acid and oligo size. Then, the dG for the oligo-target for each potential oligo is identified. (dG°_{oligo-RNA duplex}). What the disclosed data reveals is that for a given temperature there is desired requirement for this particular free energy. For example, at 37° C. the dG of oligo-target duplex should be≦about−30 kcal/mol, such as −31 kcal/mol. At 25° C. the dG should be≦about −35 kcal/mol. Furthermore, 50% of the PCR primers that are complementary to each other can be extended at 25 C if the duplex stability is −15 kcal/mol, and at 65 C if the duplex stability is only −8 kcal/mol. Thus, this thermodynamic threshold for duplex stability decreases as the temperatures decrease. Thus, as the temperature at which binding between the oligo and target decreases, the strength of the binding between the oligo and the target must increase which is consistent with there being more competing self and inter oligo structures occurring as well. Thus, after the dG of oligo-target duplex for each potential oligo is determined, a subset of oligos is identified that has less than or equal to a particular dG value, such as at 37° C. the dG should be≦about −30 kcal/mol, such as −31 kcal/mol and at 25° C. the dG should be about −35 kcal/mol. This subset of oligos can be called the oligo-target set.

33. The oligo-target set can then be analyzed, in that the dG for the self structure of each oligo in the oligo-target set and the intermolecular structure of each oligo in the oligo-target set is determined. The disclosed data indicated that there are important thermodynamic “cutoffs” that occur for each of these parameters, analogous to the thermodynamic cutoff that occurs to produce the oligo-target set of oligos. What has been identified is that for the intramolecular oligo interaction, the dG should be≧about −8 kcal/mol. The data show that this parameter changes very little between 37° C. and 25° C. For the intermolecular oligo interaction the dG should be≧about −1.1 kcal/mol. Again, the data show that this parameter changes very little between 37° C. and 25° C. For example, in certain embodiments the dG for oligo-target can be about -30. This threshold is appropriate for temperatures ranging from 25° C. to 45° C., or 28° C. to 42° C., or 32° C. to 38° C. Thus, appropriate temperatures for a dG of about −30 kcal/mol are 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C., for dGs of −30 (oligo-target), −8 (oligo-self), −1 (oligo-oligo). The optimal temperature for these thresholds is 37° C., however, at different temperatures, there is still an increase in the efficiency of the sets of oligos that are obtained for a given target. This relationship can be linear if one takes the natural log values of the values of hybridization intensity or antisense efficency.
2. Determination of dGs

34. It is understood that the method can employ any type of program for determining the dG of the various parameters, such as oligo-target, oligo-self oligo, and oligo-other oligo interactions. There are many a few free available or comercial programs which will calculate one or all of these parameters: mfold, Zipfold. M. Zuker. 2003) NucleicAcids Res. 31 (13), 3406-15, http://www.bioinfo.rpi.edu/˜-zukerm, OligoWalk (Mathews, D. H., et al., (1999), RNA, 5, 1458-1469) or OligoScreen from the package RNAstructure 3.5

http//128.151.176.70/RNAstructure.html or http://rna.chem.rochester.edu/L/),

http://www.lindenbioscience.com/pds.html (TILIA™ oligo probe design),

http://www.strandgenomics.com/SOLUTIONS/PRODUCTS/SARANI/sar_over.htm (SARANI),

http://www.mwg-biotech.com/html/d_diagnosis/d_software_oligos4array.shtml (Oligos4Array),

http://www.oligo.net/(oligo 6),

htyp://www.expresson.co.uk/services/services_5.html (ACCEssarray),

http://www.duasoftware.com (visual OMP-3) can be used.

35. For determination of dG°_{T}, all programs use thermodynamic parameters for the nearest-neighbor model (Xia, T., et al., (1998), Biochemistry, 37, 14719-14735; SantaLucia, J., Jr (1998), Proc. Natl Acad. Sci. USA, 95, 1460-1465; SantaLucia, J., Jr, et al., (1996), Biochemistry; 35, 3555-3562; Allawi, H. T. and SantaLucia, J., Jr (1997), Biochemistry, 36, 10581-10594; Sugimoto, N., et al., (1995), Biochemistry, 34, 11211-11216; Luebke, K. J., et al., (2003), Nucleic Acids Res., 31, 750-758 All of which are herein incorporated at least for material related to thermodynamic calculations).

36.Calculation of dG for Oligo-oligo self inter molecular interactions can be performed using the program OligoAnal’. (available for free downloading at http://www.gesteland.genetics.utah.edu/members/olgaM/OligAnal.ZIP. While in this general example of the method, the dG of the oligo and target for each oligo is determined before proceeding to the determination of the dG for intra and intermolecular interactions, it is understood that this is not required. For example, one could identify the dG of an oligo and target for one potential oligo, based on its value then immediately determine its intra and intermolecular dG values, and based on these results identify or discard the oligo. One could also first create an oligo-target set as described herein, and then either first identify the intramolecular oligo dG or the intermolecular oligo dG, and then identify the other. The calculations could also occur simultaneously.
3. Method for Varying Target Sequences
   a) Finding optimal hybridization oligonucleotides for varying sequences

37. As discussed herein are methods that can be used for any target sequence. However, there are a special set of target sequences, wherein the disclosed methods can be modified slightly to obtain increased efficiencies. The special set of target sequences are sequences that have varying regions. As discussed herein, for the general method, the calculations are performed, assuming that the target sequence will never change, i.e. it is always the exact sequence in all states that the oligo will bind it. This, as it turns out is a fine assumption, and even for varying sequences, the disclosed steps and parameters will provide sets of oligonucleotides with increased relative binding efficiencies. However, it is clear that there certain sequences which vary and disclosed are additional steps that can be taken, to increase the efficiency of hybridization of the set of identified oligos.

38. Identifying optimal target regions of sequences that vary is a related problem to the problem of simply identifying target regions for a single target nucleic acid. Finding optimal targets for oligonucleotides in multiple variants of related sequences is useful for a number of practical tasks. One of them is the design of oligonucleotides probes for RNA/DNA based pathogen detection assays. Beside PCR, such detection can be performed using strand displacement amplification (SDA) (Walker, G. T., et al., (1992) Nucleic Acids Res, 20, 1691-1696 and Walker, G. T., et al., (1992) Proc Natl Acad Sci USA, 89, 392-396, transcription—mediated amplification (TMA) (Kacian, D. L. and Fultz, T. J.(1995) U.S. Pat. No. 5,399,491), nucleic acid sequence-based amplification (NASBA) (Compton, J. (1991) Nature, 350, 91-92), hybridization protection assay (Arnold, L. J., Jr., et al., (1989) Clin Chem, 35, 1588-1594), branched DNA signal amplification (Urdea, M. S., et al., (1993) Aids, 7 Suppl 2, S11-14 and Urdea, M. S. (1994) Biotechnology (N Y), 12, 926-928), in situ hybridization (DeLong, E. F., et al., (1989) Science, 243, 1360-1363 and Amann, R. I., et al., (1995) Microbiol Rev, 59, 143-169) or other techniques that are currently being developed and require oligonucleotides interacting with RNA or DNA as a basic step.

39. The disclosed methods can be used to identify any nucleic acid sequence that has some variation in it. The disclosed methods, compositions, and articles, provide an approach for the combination of conservation sequence analysis with thermodynamic filtering procedures discussed herein to select optimal consensus oligonucleotide targets in multiple sequence variants, that can be used for RNA detection assays. As discussed herein, these can be performed at varying temperatures, and different results for the dG for oligo-target interactions will occur for determinations at about 37° C. to determinations at about 25° C., for example. The disclosed schemes can be used for any purpose where there is a need to eliminate RNA targets that are unlikely to interact efficiently with complementary consensus oligonucleotides where there is variation in the target sequence.

40. In general, to the filtering step discussed herein, there is added the step of forming a consensus sequence out of a set of varying sequences. This consensus sequence can be made as a separate step of the disclosed methods, or an already identified consensus sequence can be used in the disclosed methods. The disclosed data indicated that the results obtained for a consensus sequence are in agreement with the results that are obtained for a single sequence.

41. The consensus sequence can be determined using any known method as disclosed herein, as well as
b) Identification of consensus sequences

42. One aspect of the disclosed methods is the identification of a consensus sequence, for which hybridization oligonucleotides are desired. Any method of consensus sequence identification can be performed. For example, consensus sequence s for HIV-1 variants (group M) and multiple sequence alignments (Gaschen, B., et al., (2002) Bioinformatics, 17, 415-418).

43. Computer programs such as “Clustal W” (Higgins, D. G. and Sharp, P. M. (1988) Gene, 73, 237-244) http://www.ebi.ac.uk/clustalw/ for the generation of multiple sequence alignments allow detection of regions that are most conserved among many sequence variants. However, even for regions that are equally conserved, their potential utility as hybridization targets varies. Mismatches in sequence variants are more disruptive in some duplexes than in others. Additionally, the propensity for self-interactions amongst oligonucleotides targeting conserved regions differs and the structure of target regions themselves can also influence hybridization efficiency. Sequence alignments are also discussed in the section related to hybridization and sequences discussed herein.

44. In certain embodiments, calculation identifying oligos having a particular level of identity with the target region, i.e. greater than 70, 75, 80, 81, 82, 83, 84, 85, 86, 87, 88, 89, 90, 91, 92, 93, 94, 95, 96, 97, 98, or 99% can be identified. For example, once a consensus sequence is obtained, then each oligo to be analyzed as discussed herein, can first be analyzed to identify those oligos that have a minimum of a certain amount of identity with the target consensus sequence. This step, however, is not required.

45. Sensitive detection of viral RNA, such as HIV RNA, in plasma of infected persons is also achieved by methods that depend on binding of oligonucleotides to viral RNA sequences. Currently, RNA detection of some proportion of HIV-1 variants is not optimal, especially at low viral loads (Chew, C. B., et al., (1999) Aids, 13, 1977-1978 and Debyser, Z., et al., (1998) AIDS Res Hum Retroviruses, 14, 453-459) The disclosed methods, articles, and compositions allow for better HIV detection. Disclosed herein it is important to select HIV-1 RNA target regions where mutations are least disruptive for potential duplex formation with complementary oligonucleotides.

46. Optimal detection of oligonucleotide hybridization targets common to families of aligned RNA sequences requires a scheme that involves thermodynamic selection criteria. Disclosed is a scheme that addresses this and employs sequential filtering procedures. When the disclosed methods are employed against variable sequences the method typically involves first creating a consensus sequence of RNA or DNA from aligned sequence variants. Then typically the lengths of fragments to be used as oligonucleotides in the analyses are determined. Then a series of thermodynamic calculations are performed which involves selection of DNA oligonucleotides for which at least 95% of aligned sequence variants have a pairing potential greater than a defined threshold. For example, when determining the dG of the oligo-target, for a consensus sequence, rather than requiring that 100% of the oligonucleotides in the oligo-target set, have a dG of≦30 kcal/mol, but rather requiring that, for example, 95%, meet this dG threshold. This consensus factor, that could be defined as a precentage of aligned sequences that are meeting thermodynamic selection criteria can be, at least 99%, 98%, 97%, 96%, 95%, 94%, 93%, 92%, 91%, 90%, 85%, or 80. Then, a step of eliminating DNA oligonucleotides that have self-pairing potentials for intra- and/or inter-molecular interactions greater than defined thresholds occurs. Disclosed herein, this scheme has been applied to HIV-1 genomic genes and theoretically optimal RNA target regions for consensus oligonucleotides were found. The disclosed oligonucleotide probes and sets of oligonucleotide probes can be further used in oligo-probe based HIV detection techniques. The disclosed methods can be helpful in designing consensus oligonucleotides with consistent high affinity to RNA targets variants in evolutionary related genes.
4. Exemplary Target Sequences

47. There is a number of varying target sequences that can be used in the disclosed methods. For example, the target sequence can be SARS viral RNA or DNA, bacterial or fungi ribosomal RNA or DNA (5S, 16S, 18S, 25S, 28S). Practically any pathogen nucleic acid where family of related sequences can be identified and aligned.

B. Machines for Manipulation of Data and Parameters

48. It is understood that the methods disclosed herein can be performed on computers, as well as the calculations and manipulations associated with the disclosed methods. Furthermore, it is understood that the disclosed sets of primers can be manipulated, utilized, and stored on computers and computer related storage devices, such as storage media or servers.
1. Hardware

49. The hardware architecture can include a system processor potentially including multiple processing elements where each processing element may be supported via a MIPS R10000 or R4400 processor such as provided in a SILICON GRAPHICS INDIGO² IMPACT workstation. Alternative processors such as Intel-compatible processor platforms using at least one PENTIUM III or CELERON (Intel Corp., Santa Clara, Calif.) class processor, UltraSPARC (Sun Microsystems, Palo Alto, Calif.) or other equivalent processors could also be used. The system processor may include combinations of different processors from different vendors. In some embodiments, analysis and manipulation functionality, as further described below, may be distributed across multiple processing elements. The term processing element may refer to (1) a process running on a particular piece, or across particular pieces, of hardware, (2) a particular piece of hardware, or either (1) or (2) as the context allows.

50. The hardware includes a system data store (SDS) that could include a variety of primary and secondary storage elements. In one preferred embodiment, the SDS would include RAM as part of the primary storage; the amount of RAM might range from 32 MB to 640 MB or more although these amounts could vary and represent overlapping use. The primary storage may in some embodiments include other forms of memory such as cache memory, registers, non-volatile memory (e.g., FLASH, ROM, EPROM, etc.), etc.

51. The SDS may also include secondary storage including single, multiple and/or varied servers and storage elements. For example, the SDS may use internal storage devices connected to the system processor. In embodiments where a single processing element supports all of the analysis and manipulation functionality, a local hard disk drive may serve as the secondary storage of the SDS, and a disk operating system executing on such a single processing element may act as a data server receiving and servicing data requests.

52. It will be understood by those skilled in the art that the different information used in the processes and systems according to the disclosed methods may be logically or physically segregated within a single device serving as secondary storage for the SDS; multiple related data stores accessible through a unified management system, which together serve as the SDS; or multiple independent data stores individually accessible through disparate management systems, which may in some embodiments be collectively viewed as the SDS. The various storage elements that comprise the physical architecture of the SDS may be centrally located, or distributed across a variety of diverse locations. 53. The architecture of the secondary storage of the system data store may vary significantly in different embodiments. In several embodiments, database(s) may be used to store and manipulate the data; in some such embodiments, one or more relational database management systems, such as DB2 (IBM, White Plains, N.Y.), SQL Server (Microsoft, Redmond, Wash.), ACCESS (Microsoft, Redmond, Wash., ORACLE 8i (Oracle Corp., Redwood Shores, Calif.), Ingres (Computer Associates, Islandia, N.Y.), MySQL (MySQL AB, Sweden) or Adaptive Server Enterprise (Sybase Inc., Emeryville, Calif.), may be used in connection with a variety of storage devices/file servers that may include one or more standard magnetic and/or optical disk drives using any appropriate interface including, without limitation, IDE, EISA and SCSI. In some embodiments, a tape library such as Exabyte X80 (Exabyte Corporation, Boulder, Colo.), a storage attached network (SAN) solution such as available from (EMC, Inc., Hopkinton, Mass.), a network attached storage (NAS) solution such as a NetApp Filer 740 (Network Appliances, Sunnyvale, Calif.), or combinations thereof may be used.

54. In other embodiments, the data store may use database systems with other architectures such as object-oriented, spatial, object-relational or hierarchical or may use other storage implementations such as hash tables or flat files or combinations of such architectures. Such alternative approaches may use data servers other than database management systems such as a hash table look-up server, procedure and/or process and/or a flat file retrieval server, procedure and/or process. Further, the SDS may use a combination of any of such approaches in organizing its secondary storage architecture.

55. In one embodiment, coordinate data is stored in flat ASCII files according to a standardize format.

56. The hardware platform would have an appropriate operating system such as WINDOWS/NT, WINDOWS 2000 or WINDOWS/XP Server (Microsoft, Redmond, Wash.), Solaris (Sun Microsystems, Palo Alto, Calif.), or IRIX (or other UNIX/LINUX variant).
2. Data and Storage of Same

57. Data, such as sequence information or thermodynamic information, can be stored in a machine-readable form on machine-readable storage medium. Examples of such media include, but are not limited to, computer hard drive, diskette, DAT tape, CD-ROM, and the like. The information stored on this media can be used for display as a three-dimensional shape or representation thereof or for other uses based on the structural coordinates, the spatial relationships between atoms described by the structural coordinates or the three-dimensional structures that they define or for analysis of the thermodynamic parameters discussed herein. Such uses can include the use of a computer capable of reading the data from the storage media and executing instructions to generate and/or manipulate structures defined by the data.
3. Machine Readable Storage Media

58. Disclosed are machine-readable storage mediums comprising a data storage material encoded with machine readable data. Furthermore, the data can be extracted and manipulated by machines configured to read the data stored on the machine readable storage media, and in fact, when performing the thermodynamic calculations, as discussed herein, typically the data will be retrieved or stored on a machine readable storage media.

59. The disclosed coordinates and data can be manipulated on any appropriate machine, having for example, a processor, memory, and a monitor. The data can also be manipulated and accessed by a variety of connected items, including printers, LCDs, for example.

60. It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

C. Compositions

61. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular HIV GAG probe is disclosed and discussed and a number of modifications that can be made to a number of molecules including the HIV GAG probe are discussed, specifically contemplated is each and every combination and permutation of HIV GAG probe and the modifications that are possible unless specifically indicated to the contrary. Thus, if a class of molecules A, B, and C are disclosed as well as a class of molecules D, E, and F and an example of a combination molecule, A—D is disclosed, then even if each is not individually recited each is individually and collectively contemplated meaning combinations, A—E, A—F, B—D, B—E, B—F, C—D, C—E, and C—F are considered disclosed. Likewise, any subset or combination of these is also disclosed. Thus, for example, the sub-group of A—E, B—F, and C—E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.
1. Preferred Primer
   a) Viral

62. FIG. 14 shows a plot of the oligonucleotides meeting the requirements outlined herein. These oligonucleotides as various disclosed sets can be used in DNA chips, as antisense molecules, and as diagnostic probes, for example. It is understood that any virus can be a target and that the sequences for these viruses can be found at Genbank and are herein incorporated by reference in their entirety. Furthermore, for any virus, the sequence can be obtained using standard techniques.

63. Viruses that are suitable for the methods and uses described herein can include both DNA viruses and RNA viruses. Exemplary viruses can belong to the following none exclusive list of families Adenoviridae, Arenaviridae, Astroviridae, Baculoviridae, Barnaviridae, Betaherpesvirinae, Bimaviridae, Bromoviridae, Bunyaviridae, Caliciviridae, Chordopoxvirinae, Circoviridae, Comoviridae, Coronaviridae, Cystoviridae, Corticoviridae, Entomopoxvirinae, Filoviridae, Flaviviridae, Fuselloviridae, Geminiviridae, Hepadnaviridae, Herpesviridae, Gammaherpesvirinae, Inoviridae, Iridoviridae, Leviviridae, Lipothrixviridae, Microviridae, Myoviridae, Nodaviridae, Orthomyxoviridae, Papovaviridae, Paramyxoviridae, Paramyxovirinae, Partitiviridae, Parvoviridae, Phycodnaviridae, Picomaviridae, Plasmaviridae, Pneumovirinae, Podoviridae, Polydnaviridae, Potyviridae, Poxviridae, Reoviridae, Retroviridae, Rhabdoviridae, Sequiviridae, Siphoviridae, Tectiviridae, Tetraviridae, Togaviridae, Tombusviridae, and Totiviridae.

64. Specific examples of suitable viruses include, but are not limited to, Mastadenovirus, Human adenovirus 2, Aviadenovirus, African swine fever virus, arenavirus, Lymphocytic choriomeningitis virus, Ippy virus, Lassa virus, Arterivirus, Human astrovirus 1, Nucleopolyhedrovirus, Autographa californica nucleopolyhedrovirus, Granulovirus, Plodia interpunctella granulovirus, Badnavirus, Commelina yellow mottle virus, Rice tungro bacilliform, Barnavirus, Mushroom bacilliform virus, Aquabirnavirus, Infectious pancreatic necrosis virus, Avibirnavirus, Infectious bursal disease virus, Entomobirnavirus, Drosphilia X virus, Alfamovirus, Alfalfa mosaic virus, Ilarvirus, Ilarvirus Subgroups 1-10, Tobacco streak virus, Bromovirus, Brome mosaic virus, Cucumovirus, Cucumber mosaic virus, Bhanja virus Group, Kaisodi virus, Mapputta virus, Okola virus, Resistencia virus, Upolu virus, Yogue virus, Bunyavirus, Anopheles A virus, Anopheles B virus, Bakau virus, Bunyamwera virus, Bwamba virus, C virus, California encephalitis virus, Capim virus, Gamboa virus, Guama virus, Koongol virus, Minatitlan virus, Nyando virus, Olifantsvlei virus, Patois virus, Simbu virus, Tete virus, Turlock virus, Hantavirus, Hantaan virus, Nairovirus, Crimean-Congo hemorrhagic fever virus, Dera Ghazi Khan virus, Hughes virus, Nairobi sheep disease virus, Qalyub virus, Sakhalin virus, Thiafora virus, Crimean-congo hemorrhagic fever virus, Phlebovirus, Sandfly fever virus, Bujaru complex, Candiru complex, Chilibre complex, Frijoles complex, Punta Toro complex, Rift Valley fever complex, Salehabad complex, Sandfly fever Sicilian virus, Uukuniemi virus, Uukuniemi virus, Tospovirus, Tomato spotted wilt virus, Calicivirus, Vesicular exanthema of swine virus, Capillovirus, Apple stem grooving virus, Carlavirus, Carnation latent virus, Caulimovirus, Cauliflower mosaic virus, Circovirus, Chicken anemia virus, Closterovirus, Beet yellows virus, Comovirus, Cowpea mosaic virus, Fabavirus, Broad bean wilt virus 1, Nepovirus, Tobacco ringspot virus, Coronavirus, Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, Rat coronavirus, Turkey coronavirus, Rabbit coronavirus, Torovirus, Berne virus, Breda virus, Corticovirus, Alteromonas phage PM2, Pseudomonas Phage phi6, Deltavirus, Hepatitis delta virus, Dianthovirus Carnation ringspot virus, Red clover necrotic mosaic virus, Sweet clover necrotic mosaic virus, Enamovirus, Pea enation mosaic virus, Filovirus, Marburg virus, Ebola virus Zaire, Flavivirus, Yellow fever virus, Tick-borne encephalitis virus, Rio Bravo Group, Japanese encephalitis, Tyuleniy Group, Ntaya Group, Uganda S Group, Dengue Group, Modoc Group, Pestivirus, Bovine diarrhea virus, Hepatitis C virus, Furovirus, Soil-borne wheat mosaic virus, Beet necrotic yellow vein virus, Fusellovirus, Sulfobolus virus 1, Subgroup I, II, and III geminivirus, Maize streak virus, Beet curly top virus, Bean golden mosaic virus, Orthohepadnavirus, Hepatitis B virus, Avihepadnavirus, Alphaherpesvirinae, Simplexvirus, Human herpesvirus 1, Varicellovirus, Human herpesvirus 3, Cytomegalovirus, Human herpesvirus 5, Muromegalovirus, Mouse cytomegalovirus 1, Roseolovirus, Human herpesvirus 6, Lymphocryptovirus, Human herpesvirus 4, Rhadinovirus, Ateline herpesvirus 2, Hordeivirus, Barley stripe mosaic virus, Hypoviridae, Hypovirus, Cryphonectria hypovirus 1-EP713, Idaeovirus, Raspberry bushy dwarf virus, Inovirus, Coliphage fd, Plectrovirus, Acholeplasma phage L51, Iridovirus, Chilo iridescent virus, Chloriridovirus, Mosquito iridescent virus, Ranavirus, Frog virus 3, Lymphocystivirus, Lymphocystis disease virus flounder isolate, Goldfish virus 1, Levivirus, Enterobacteria phage MS2, Allolevirus, Enterobacteria phage Qbeta, Lipothrixvirus, Thermoproteus virus 1, Luteovirus, Barley yellow dwarf virus, Machlomovirus, Maize chlorotic mottle virus, Marafivirus, Maize rayado fino virus, Microvirus, Coliphage phiX174, Spiromicrovirus, Spiroplasma phage 4, Bdellomicrovirus, Bdellovibrio phage MAC 1, Chlamydiamicrovirus, Chlamydia phage 1, T4-like phages, coliphage T4, Necrovirus, Tobacco necrosis virus, Nodavirus, Nodamura virus, Influenzavirus A, B and C, Thogoto virus, Polyomavirus, Murine polyomavirus, Papillomavirus, Rabbit (Shope) Papillomavirus, Paramyxovirus, Human parainfluenza virus 1, Morbillivirus, Measles virus, Rubulavirus, Mumps virus, Pneumovirus, Human respiratory syncytial virus, Partitivirus, Gaeumannomyces graminis virus 019/6-A, Chrysovirus, Penicillium chrysogenum virus, Alphacryptovirus, White clover cryptic viruses 1 and 2, Betacryptovirus, Parvovirinae, Parvovirus, Minute mice virus, Erythrovirus, B19 virus, Dependovirus, Adeno-associated virus 1, Densovirinae, Densovirus, Junonia coenia densovirus, Iteravirus, Bombyx mori virus, Contravirus, Aedes aegypti densovirus, Phycodnavirus, 1-Paramecium bursaria Chlorella NC64A virus group, Paramecium bursaria chlorella virus 1,2-Paramecium bursaria Chlorella Pbi virus, 3-Hydra viridis Chlorella virus, Enterovirus, Human poliovirus 1, Rhinovirus Human rhinovirus 1A, Hepatovirus, Human hepatitis A virus, Cardiovirus, Encephalomyocarditis virus, Aphthovirus, Foot-and-mouth disease virus, Plasmavirus, Acholeplasma phage L2, Podovirus, Coliphage T7, Ichnovirus, Campoletis sonorensis virus, Bracovirus, Cotesia melanoscela virus, Potexvirus, Potato virus X, Potyvirus, Potato virus Y, Rymovirus, Ryegrass mosaic virus, Bymovirus, Barley yellow mosaic virus, Orthopoxvirus, Vaccinia virus, Parapoxvirus, Orf virus, Avipoxvirus, Fowlpox virus, Capripoxvirus, Sheep pox virus, Leporipoxvirus, Myxoma virus, Suipoxvirus, Swinepox virus, Molluscipoxvirus, Molluscum contagiosum virus, Yatapoxvirus, Yaba monkey tumor virus, Entomopoxviruses A, B, and C, Melolontha melolontha entomopoxvirus, Amsacta moorei entomopoxvirus, Chironomus luridus entomopoxvirus, Orthoreovirus, Mammalian orthoreoviruses, reovirus 3, Avian orthoreoviruses, Orbivirus, African horse sickness viruses 1, Bluetongue viruses 1, Changuinola virus, Corriparta virus, Epizootic hemarrhogic disease virus 1, Equine encephalosis virus, Eubenangee virus group, Lebombo virus, Orungo virus, Palyam virus, Umatilla virus, Wallal virus, Warrego virus, Kemerovo virus, Rotavirus, Groups A-F rotaviruses, Simian rotavirus SA11, Coltivirus, Colorado tick fever virus, Aquareovirus, Groups A-E aquareoviruses, Golden shiner virus, Cypovirus, Cypovirus types 1-12, Bombyx mori cypovirus 1, Fijivirus, Fijivirus groups 1-3, Fiji disease virus, Fijivirus groups 2-3, Phytoreovirus, Wound tumor virus, Oryzavirus, Rice ragged stunt, Mammalian type B retroviruses, Mouse mammary tumor virus, Mammalian type C retroviruses, Murine Leukemia Virus, Reptilian type C oncovirus, Viper retrovirus, Reticuloendotheliosis virus, Avian type C retroviruses, Avian leukosis virus, Type D Retroviruses, Mason-Pfizer monkey virus, BLV-HTLV retroviruses, Bovine leukemia virus, Lentivirus, Bovine lentivirus, Bovine immunodeficiency virus, Equine lentivirus, Equine infectious anemia virus, Feline lentivirus, Feline immunodeficiency virus, Canine immunodeficiency virus Ovine/caprine lentivirus, Caprine arthritis encephalitis virus, Visna/maedi virus, Primate lentivirus group, Human immunodeficiency virus 1, Human immunodeficiency virus 2, Human immunodeficiency virus 3, Simian immunodeficiency virus, Spumavirus, Human spuma virus, Vesiculovirus, Vesicular stomatitis Indiana virus, Lyssavirus, Rabies virus, Ephemerovirus, Bovine ephemeral fever virus, Cytorhabdovirus, Lettuce necrotic yellows virus, Nucleorhabdovirus, Potato yellow dwarf virus, Rhizidiovirus, Rhizidiomyces virus, Sequivirus, Parsnip yellow fleck virus, Waikavirus, Rice tungro spherical virus, Lambda-like phages, Coliphage lambda, Sobemovirus, Southern bean mosaic virus, Tectivirus, Enterobacteria phage PRD1, Tenuivirus, Rice stripe virus, Nudaurelia capensis beta-like viruses, Nudaurelia beta virus, Nudaurelia capensis omega-like viruses, Nudaurelia omega virus, Tobamovirus, Tobacco mosaic virus (vulgare strain; ssp. NC82 strain), Tobravirus, Tobacco rattle virus, Alphavirus, Sindbis virus, Rubivirus, Rubella virus, Tombusvirus, Tomato bushy stunt, virus, Carmovirus, Carnation mottle virus, Turnip crinkle virus, Totivirus, Saccharomyces cerevisiae virus, Giardiavirus, Giardia lamblia virus, Leishmaniavirus, Leishmania brasiliensis virus 1-1, Trichovirus, Apple chlorotic leaf spot virus, Tymovirus, Turnip yellow mosaic virus, Umbravirus, and Carrot mottle virus.
b) Bacteria

65. Any type of bacteria nucleic acid can also be a target. Examples of bacterium nucleic acid include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Sinikania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholerae, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The sequences for the genomes of these bacteria exist at Genbank and can be identified using routine molecular techniques for sequencing nucleic acid.
c) Parasites

66. The disclosed methods can also be used against any parasite. Examples of parasites include, but are not limited to, Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica, or any strain or variant thereof. The sequences for the genomes of these parasites exist at Genbank and can be identified using routine molecular techniques for sequencing nucleic acid.
d) Fungi

67. The disclosed methods can also be used against any fungi. Examples of fungi include, but are not limited to, Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternate, and variations or different strains of these. The sequences for the genomes of these parasites exist at Genbank and can be identified using routine molecular techniques for sequencing nucleic acid.
2. Sequence Similarities

68. It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

69. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent of identity or similarity of every alighned symbol, which could be nucleotide or amino-acid . Those of skill in the art readily understand how to evaluate homology of two proteins or nucleic acids, such as genes. For example, the homology can be calculated after aligning the two sequences so that the homology is at its highest level.

70. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

71. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

72. For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).
3. Hybridization/Selective Hybridization

73. The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

74. Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G—C or A—T richness of any area wherein variability is searched for. Likewise, stringency of hybridization and washing, if desired, can be increased accordingly as homology desired is increased, and further, depending upon the G—C or A—T richness of any area wherein high homology is desired, all as known in the art.

75. Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

76. Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

77. Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

78. It is understood that those of skill in the art understand that if a composition or method meets any one of these criteria for determining hybridization either collectively or singly it is a composition or method that is disclosed herein.
a) Examples of molecules that can be designed using the disclosed methods, compositions, and articles.
   (1) Primers and probes

79. Disclosed are compositions including primers and probes, which are capable of interacting with the genes disclosed herein. In certain embodiments the primers are used to support DNA, RNA or signal amplification reactions. Typically the primers will be capable of being extended in a sequence specific manner. Alktemativly oligo-probes can be used to amplify the nucleic acid sequence specific signal. The examples include in situ oligo-target hybridization (DeLong, E. F., et al., (1989) Science, 243, 1360-1363 and Amann, R. I., et al., (1995) Microbiol Rev, 59, 143-169) or branch DNA signal amplification technology (Urdea, M. S., et al., (1993) Aids, 7 Suppl 2, S11-14 and Urdea, M. S. (1994) Biotechnology (N.Y.), 12, 926-928), Extension of a primer or signal amplification in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription in situ hybridization and branch DNA signal amplification. Techniques and conditions that amplify the primer or signal in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

80. The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

81. In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

82. The primers for the HIV-1 genomic DNA or RNA, such GAG RNA, for example, typically will be used to produce an amplified DNA product or signal for a region of the HIV genome. In general, typically the size of the product will be such that the size can be accurately determined to within 3, or 2 or 1 nucleotides.

83. In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

84. In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.
(2) Functional Nucleic Acids

85. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

86. Functional nucleic acid molecules can interact with any macromolecule, such as DNA, RNA, polypeptides, or carbohydrate chains. Thus, functional nucleic acids can interact with the mRNA of HIV genomic RNA, for example, such as GAG RNA, or the genomic DNA of HIV genomic RNA, for example, such as GAG DNA or they can interact with the polypeptide of the HIV genome, for example, such as the GAG polypeptide, for example. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

87. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos.: 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

88. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of HIV aptamers, for example, such as GAG aptamers, for example, the background protein could be serum albumin. Representative examples of how to make and use aptamers to bind a variety of different target molecules can be found in the following non-limiting list of U.S. Pat. Nos.: 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

89. Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos.: 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos.: 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos.: 5,595,873 and 5,652,107). There are also a number of ribozymes that are not found in natural systems, but which have been engineered to catalyze specific reactions de novo (for example, but not limited to the following U.S. Pat. Nos.: 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos.: 5,646,042, 5,693,535, 5,731,295, 5,811,300, 5,837,855, 5,869,253, 5,877,021, 5,877,022, 5,972,699, 5,972,704, 5,989,906, and 6,017,756.

90. Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻². Representative examples of how to make and use triplex forming molecules to bind a variety of different target molecules can be found in the following non-limiting list of U.S. Pat. Nos.: 5,176,996, 5,645,985, 5,650,316, 5,683,874, 5,693,773, 5,834,185, 5,869,246, 5,874,566, and 5,962,426.

91. External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

92. Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos.: 5,168,053, 5,624,824, 5,683,873, 5,728,521, 5,869,248, and 5,877,162.
4. Nucleic Acids

93. There are a variety of molecules disclosed herein that are nucleic acid based, including for example the nucleic acids that encode, for example HIV proteins, such as GAG, or any of the nucleic acids disclosed herein for making functional knockouts, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed MRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.
a) Nucleotides and related molecules

94. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

95. A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

96. Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conforn to a double helix type structure when interacting with the appropriate target nucleic acid. There are many varieties of these types of molecules available in the art and available herein.

97. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556). There are many varieties of these types of molecules available in the art and available herein.

98. A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, Ni, and C6 positions of a purine based nucleotide, nucleotide analog, or nucleotide substitute and the C2, N3, C4 positions of a pyrimidine based nucleotide, nucleotide analog, or nucleotide substitute.

99. A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.
b) Sequences

100. There are a variety of sequences related to the protein molecules disclosed herein, for example, nucleic acids related to the HIV genome, such as HIV GAG, or any of the nucleic acids disclosed herein for making HIV GAG, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.
5. Nucleic Acid Delivery

101. In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

102. As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

103. As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

104. Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.
6. Pharmaceutical Carriers/Delivery of Pharmaceutical Products

105. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

106. The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

107. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

108. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Imunol. Immunother., 35:421-425, (1992); Pietersz and McKenzie, Imnmunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).
a) Pharmaceutically Acceptable Carriers

109. The compositions, including antibodies, can be used therapeutically in combination with a pharmaceutically acceptable carrier.

110. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

111. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

112. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

113. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

114. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

115. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

116. Compositions for oral administration include powders or granules, suspensions or solutions in water or non-aqueous media, capsules, sachets, or tablets. Thickeners, flavorings, diluents, emulsifiers, dispersing aids or binders may be desirable.

117. Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.
b) Therapeutic Uses

118. Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

119. Following administration of a disclosed composition, such as an antisense molecule, for treating, inhibiting, or preventing an HIV infection, the efficacy of the therapeutic antisense molecule can be assessed in various ways well known to the skilled practitioner. For instance, one of ordinary skill in the art will understand that a composition, such as an antibody, disclosed herein is efficacious in treating or inhibiting an HIV infection in a subject by observing that the composition reduces viral load or prevents a further increase in viral load. Viral loads can be measured by methods that are known in the art, for example, using polymerase chain reaction assays to detect the presence of HIV nucleic acid or antibody assays to detect the presence of HIV protein in a sample (e.g., but not limited to, blood) from a subject or patient, or by measuring the level of circulating anti-HIV antibody levels in the patient. Efficacy of the administration of the disclosed composition may also be determined by measuring the number of CD4+ T cells in the HIV-infected subject. An antibody treatment that inhibits an initial or further decrease in CD4+ T cells in an HIV-positive subject or patient, or that results in an increase in the number of CD4+ T cells in the HIV-positive subject, is an efficacious antibody treatment.

120. The compositions that inhibit interactions disclosed herein may be administered prophylactically to patients or subjects who are at risk for being exposed to HIV or who have been newly exposed to HIV. In subjects who have been newly exposed to HIV but who have not yet displayed the presence of the virus (as measured by PCR or other assays for detecting the virus) in blood or other body fluid, efficacious treatment with an antibody partially or completely inhibits the appearance of the virus in the blood or other body fluid.
7. Chips and micro arrays

121. Disclosed are chips where at least one address is the sequences or part of the sequences set forth in any of the nucleic acid sequences or sets of nucleic acids disclosed herein. Also disclosed are chips where at least one address is the sequences or portion of sequences set forth in any of the peptide sequences or sets of peptide sequences disclosed herein.

122. Also disclosed are chips where at least one address is a variant of the sequences or part of the sequences set forth in any of the nucleic acid sequences or sets of nucleic acids disclosed herein. Also disclosed are chips where at least one address is a variant of the sequences or portion of sequences set forth in any of the peptide sequences or sets of peptides disclosed herein.
8. Kits

123. Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for determining whether a subject has an HIV infection, comprising the oligonucleotides set forth in for example FIG. 14.

D. Methods of making the compositions

124. The compositions disclosed herein and the compositions necessary to perform the disclosed methods can be made using any method known to those of skill in the art for that particular reagent or compound unless otherwise specifically noted.
1. Nucleic acid synthesis

125. For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymzol., 65:610-620 (1980), (phosphotriester method). Protein nucleic acid molecules can be made using known methods such as those described by Nielsen et al., Bioconjug. Chem. 5:3-7 (1994).
2. Process claims for making the compositions

126. Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

127. Disclosed are nucleic acid molecules produced by the process comprising linking in an operative way a nucleic acid comprising the sequence set forth in herein and a sequence controlling the expression of the nucleic acid.

128. Also disclosed are nucleic acid molecules produced by the process comprising linking in an operative way a nucleic acid molecule comprising a sequence having 80% identity to a sequence set forth in herein, and a sequence controlling the expression of the nucleic acid.

129. Disclosed are nucleic acid molecules produced by the process comprising linking in an operative way a nucleic acid molecule comprising a sequence that hybridizes under stringent hybridization conditions to a sequence set forth herein and a sequence controlling the expression of the nucleic acid.

130. Disclosed are nucleic acid molecules produced by the process comprising linking in an operative way a nucleic acid molecule comprising a sequence encoding a peptide set forth in herein and a sequence controlling an expression of the nucleic acid molecule.

131. Disclosed are nucleic acid molecules produced by the process comprising linking in an operative way a nucleic acid molecule comprising a sequence encoding a peptide having 80% identity to a peptide set forth in herein and a sequence controlling an expression of the nucleic acid molecule.

132. Disclosed are nucleic acids produced by the process comprising linking in an operative way a nucleic acid molecule comprising a sequence encoding a peptide having 80% identity to a peptide set forth in herein, wherein any change from the herein are conservative changes and a sequence controlling an expression of the nucleic acid molecule.

133. Disclosed are cells produced by the process of transforming the cell with any of the disclosed nucleic acids. Disclosed are cells produced by the process of transforming the cell with any of the non-naturally occurring disclosed nucleic acids.

134. Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

135. Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

136. Also disclose are animals produced by the process of adding to the animal any of the cells disclosed herein.

E. Methods of using the compositions
1. Methods of using the compositions as research tools

137. The disclosed compositions can be used in a variety of ways as research tools. For example, the disclosed compositions, such as the disclosed sequences can be used to study the structure of the target nucleic acids.

138. The compositions can be used for example as targets in combinatorial chemistry protocols or other screening protocols to isolate molecules that possess desired functional properties related to, for example, antisense molecules.

139. The disclosed compositions can also be used diagnostic tools related to diseases HIV and other viral or bacteria or pathogens.

140. The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis of for example, HIV, particularly allelic analysis as it relates to different strains. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

F. Terms

141. As used in the specification and the appended claims, the singular forms “a, ” “an” and “the” include plural referents unless the context clearly dictates otherwise. Thus, for example, reference to “a pharmaceutical carrier” includes mixtures of two or more such carriers, and the like.

142. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

143. In this specification and in the claims which follow, reference will be made to a number of terms which shall be defined to have the following meanings:

144. “Optional” or “optionally” means that the subsequently described event or circumstance may or may not occur, and that the description includes instances where said event or circumstance occurs and instances where it does not.

145. “Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

146. “Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

147. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

148. The present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### G. EXAMPLES

149. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in °C. or is at ambient temperature, and pressure is at or near atmospheric.

### 1. Example 1 Identification of Optimal Oligo Target Regions and Oligos: Thermodynamic Calculations and Statistical Correlations for Oligo-probes Design

a) Materials and Methods
   (1) Oligonucleotide datasets of hybridization experiments

150. Three experimental datasets were used for statistical analysis. For obtaining dataset 1, Affymetrix GeneChip.TM.HIV PRT produced by Affymetrix Corporation, Santa Clara, Calif. was used. For obtaining datasets 2 and 3, a chip produced by Oxford Gene Technology, Oxford, UK was used. For all datasets, in vitro transcribed non-fragmented HIV-1 RNA was used for the hybridization experiments. The hybridization intensities of oligo probes targeting every overlapping 20 nucleotide fragments of the relevant RNA were collected for dataset 1. The hybridization intensities of oligo-probes targeting every overlapping 20 nucleotide fragments and every 21 nucleotide fragments of the relevant RNA were collected for dataset 2. The hybridization intensities of oligo-probes targeting every overlapping nucleotide fragment ranging in size from 3 to 21 nucleotides of the relevant RNA were collected for dataset 3. The experiments were performed with oligonucleotides immobilized on a solid support. The experimental conditions used to obtain the datasets are given in Table 1.

TABLE 1
Summary of differences and similarities between hybridization
experiments that were performed to obtain the datasets

| | Dataset 1 | Dataset 2 | Dataset 3 |
|---|---|---|---|
| Target RNA length | 1041 nt | 290 nt | 290 nt |
| Temperature of hybridization | 37° C. | 25° C. | 25° C. |
| Length of the oligo-probe | 20 nt | 20 and 21 nt | 3-21 nt |
| RNA target labeled with | fluorescein | P³³ | P³³ |
| Concentration of target RNA in | 26.3 nM | 2.5 nM | 2.5 nM |
| experiment | | | |
| Number of experimental data | 1021 | 541 | 6156 |
| points in the dataset | | | |

(2) Thermodynamic calculations

152. Calculations of thermodynamic properties of oligonucleotides were done with the help of newly created and pre-existing software. For the oligonucleotides that were involved in the experiments performed at 37° C., the program OligoWalk from the package RNA structure 3.7 was used (Mathews, D. H., et al., (1999), RNA, 5, 1458-1469) (http://128.151.176.70/RNAstricture.html). For the oligonucleotides that were involved in the experiments performed at 25° C., Excel macro ‘OligoAnal’ was created (available for downloading at http://www.gesteland.genetics.utah.edu/members/olgaM/OligAnal.ZIP). Using thermodynamic parameters for the nearest neighbor model (SantaLucia, J., Jr, et al., (1996), Biochemistry, 35, 3555-3562; Allawi, H. T. and SantaLucia, J., Jr (1997), Biochemistry, 36, 10581-10594; Allawi, H. T. and SantaLucia, J., Jr (1998), Nucleic Acids Res., 26, 2694-2701; Allawi, H. T. and SantaLucia, J., Jr (1998), Biochemistry, 37, 2170-2179; Allawi, H. T. and SantaLucia, J., Jr (1998), Biochemistry, 37, 9435-9444; Peyret, N., et al., (1999), Biochemistry, 38, 3468-3477; SantaLucia, J., Jr (1998), Proc. Natl Acad. Sci. USA, 95, 1460-1465; Sugimoto, N., et al., (1995), Biochemistry, 34, 11211-11216), this macro can produce relevant dG°_{T} values (oligonucleotide inter-molecular and oligo-target pairing potentials) for each analyzed oligonucleotide. For calculation of oligonucleotide intra-molecular pairing potentials at 25° C., the program mfold version 3.0 (http://www.bioinfo.rpi.edu/applications/mfold/old/rna/form4.cgi) with thermodynamic parameters from the version 3.1 was used (SantaLucia, J., Jr (1998), Proc. Natl Acad. Sci. USA, 95, 1460-1465) (http://www.bioinfo.rpi.edu/zukerm/dna/credit.html). Nucleic acid conformation was assumed to be linear and the ionic conditions were set at 1 M Na+. In the program output, the positive values of dG°₂₅ were changed to 0.
(3) Statistical analysis

153. Statistical tools from Excel (Microsoft, Inc.) were used for correlation analysis (t-test) and scatter-plot data presentations. The oligonucleotides in both datasets were categorized into groups according to their hybridization intensity. Two thresholds for oligonucleotide categorizations were created: the upper threshold and the lower threshold. In both datasets the thresholds were set identically. The upper thresholds for logarithmic values of RNA hybridization intensity were set as 9, the lower thresholds for logarithmic values of RNA hybridization intensity were set as 8.
(4) Thermodynamic filtration

154. The process of selection of oligo-probe sets using several thermodynamic criteria was called thermodynamic filtration.
b) Results

155. A schematic illustration of the competing molecular interactions relevant to oligo-RNA binding is shown in FIG. 1. To estimate how thermodynamic evaluations of the stability of an RNA-DNA duplex and the stability of oligonucleotide self-structures can be related to oligonucleotide RNA binding properties, two datasets of hybridization experiments performed with oligonucleotide scanning arrays were analyzed.

156. Data for the first set were taken from the literature (Shannon, K. and Wolber, P. (2002) Method for evaluating oligonucleotide probe sequences. U.S. Pat. No. 6,251,588), while data for the second set were kindly provided by Dr Verhoef from Oxford Gene Technology. The differences and similarities between the two hybridization experiments that were performed to obtain the two datasets are summarized in Table 1 (see also Materials and Methods).

157. The results of the oligonucleotide scanning array hybridization experiment that were used for creation of dataset 1 are presented graphically in FIG. 2. A sharp contrast is evident between different oligonucleotides in their ability to hybridize with target RNA. By statistical analysis, it was explored if this hybridization intensity contrast can be related to oligonucleotide thermodynamic properties.

158. dG°_{T} values for competing molecular interactions relevant to oligo-RNA binding were calculated for each oligonucleotide in the datasets based on thermodynamic parameters of the nearest neighbor model (see thermodynamic calculations in Materials and Methods). Correlation analyses (t-tests) of both datasets were performed (Table 2). For datasets 1 and 2, significant correlations (P<0.01) were detected between the experimental hybridization intensity and the theoretical dG°_{T} values associated with stability of oligonucleotide self-structures and oligonucleotide-RNA duplexes.

TABLE 2
Correlations between thermodynamic properties of oligonucleotides and
their experimental RNA affinity

| correlation coefficients for absolute values | Dataset 1 | Dataset 2 |
|---|---|---|
| ΔG°_{T} oligo duplex with RNA versus | 0.46 | 0.30 |
| ln(hybridization intensity) | | |
| ΔG°_{T} oligo intra-molecular structure | −0.28 | −0.52 |
| versus ln(hybridization intensity) | | |
| ΔG°_{T} oligo inter-molecular structure | −0.2 | −0.40 |
| versus ln(hybridization intensity) | | |

160.

161. Scatter plots (FIG. 3) illustrate the relationship between the experimental intensity of hybridization signals and thermodynamic properties of oligonucleotides from the two datasets. Since the slope of the trend line in scatter plots indicates the existence of a correlation between two variables, a positive correlation is evident between the absolute value of the thermodynamic evaluation of oligonucleotide-RNA duplex stability and intensity of DNA-RNA hybridization (FIG. 3, top plots). In contrast, the slopes of the trend lines indicate that there is a negative correlation between the absolute dG°_{T} values of oligonucleotide self-pairing and the intensity of DNA-RNA hybridization (FIG. 3, middle and bottom plots). An attempt to adjust mfold program input to improve evaluation of oligonucleotide intra-molecular self-structure by changing sodium or magnesium concentrations was not successful. Surprisingly, even though the experiments were performed at 100 mM Na+, the best correlations between theoretical and experimental values were achieved when the ionic conditions in the program input were set at 1 M Na+.

162. The existence of a significant correlation between mfold calculated dG°_{T} values of oligonucleotide self-pairing and the intensity of DNA-RNA hybridization indicates that mfold can be employed for the prediction of stability of oligo probe self-structures. The current version of mfold complies nearest-neighbor as well as hairpin, bulge, internal and multi-branched loop parameters from different sources (http://www.bioinfo.rpi.edu/zukerm/dna/credit.html). Perhaps thermodynamic parameters derived from one reliable modem source would be better. Obtaining optimized thermodynamic parameters can likely lead to a significant improvement of mfold prediction performance.

163. The next issue is how to employ the statistical findings described herein and how to find thermodynamic thresholds for selection of oligonucleotide sets with a high proportion of efficient RNA binders. Variable, arbitrarily chosen cut-off points for all three thermodynamic criteria were applied, and the proportions of efficient RNA binders in the filtered oligo subset were determined for each combination. A combination that delivered the oligo subset with a high proportion of efficient RNA binders was found. Experimental data can also be used for statistical analysis, for example, using rational weighting of each thermodynamic parameter employing an equation suggested in Mathews (Mathews, D. H., et al., (1999), RNA, 5, 1458-1469).

164. In this study, the oligonucleotides in both datasets were categorized into groups according to the experimental intensity of DNA-RNA hybridization using certain arbitrarily chosen thresholds as described in the Materials and Methods (FIG. 2). The group of efficient RNA binders includes oligonucleotides with DNA-RNA hybridization intensity higher than the upper threshold. The group of poor binders includes oligonucleotides with values worse than the lower threshold. Finally, the group of intermediate binders includes oligonucleotides with DNA-RNA hybridization intensity between the two thresholds.

165. The proportions of efficient RNA binders among oligonucleotides were calculated in both datasets (FIG. 4). These proportions were also calculated for the probe subsets that were created using only oligonucleotides with certain thermodynamic properties. The proportions of efficient RNA binders were larger in the subsets that were predicted to form more stable oligonucleotide-RNA duplexes in comparison with the datasets of all probes (FIG. 4). These proportions become even larger if oligonucleotides that are able to form self-structures of specified stability are excluded (FIG. 4). The process of selection of oligo-probe sets using several thermodynamic criteria can deliver a high proportion of efficient RNA binders. Disclosed herein this process can be called thermodynamic filtration.

166. It is interesting that filtering out of the oligonucleotides that form intermolecular structures of specified stability increases the proportion of efficient RNA binders. It likely indicates that oligo-oligo intermolecular interaction can occur during hybridization experiments even though the oligonucleotides are covalently attached through their ends to a solid support.

167. Both thermodynamic evaluations of oligonucleotide intra- and inter-molecular self-interacting properties are strongly correlated to each other. The steep slopes of the trend lines of both scatter plots (FIG. 5), and highly significant correlation coefficients (0.54 for the first dataset and 0.66 for the second dataset, p<0.001) demonstrate this point. Sometimes, if two variables are highly correlated, only one is sufficient for predictive purposes. However, it was found that both thermodynamic criteria for self-structure forming potentials are simultaneously useful for efficient discrimination into subsets that mainly contain efficient or poor RNA binders (FIG. 5).

168. Disclosed herein is the analysis of experimental datasets that combine hybridization data for two different RNAs. The temperature used for the hybridization experiments that yielded dataset 1 was 37° C., and for datasets 2 and 3, it was 25° C. For the subsets with the highest proportion of efficient RNA binders, the filtration (dG°_{T}) cut-offs for DNA-RNA duplex stability are different; −35 kcal/mol for the experiments that were performed at 25° C. and −29 kcal/mol for the experiments that were performed at 37° C. (FIGS. 4 and 6). Temperature, concentration of target RNA, and ionic conditions of hybridization are the factors that can influence optimal filtration cut-off points. This work, however, demonstrates that, regardless of differences in the experimental conditions, thermodynamic filtration involving criteria of oligo-RNA duplex and oligo self-structure stabilities can be helpful for efficient elimination of poor RNA binders.

169. Correlations between thermodynamic factors and experimental binding of oligonucleotides with RNA or DNA targets were found previously (Mathews, D. H., et al., (1999), RNA, 5, 1458-1469, Walton, S. P., et al., (1999), Biotechnol. Bioeng., 65, 1-9; Jayaraman, A., et al., (2002), Biochim. Biophys. Acta, 1520, 105-114; Walton, S. P., et al., (2002), Biophys. J., 82, 366-388; Luebke, K. J., et al., (2003), Nucleic Acids Res., 31, 750-758). Disclosed herein is that selection of oligonucleotides using a thermodynamic filtration approach can increase, by several-fold, the proportion of DNA oligonucleotides that can bind RNA efficiently. For gene expression monitoring with the DNA chips, a similar approach can minimize the number of oligo-probes needed per gene, thereby increasing the number of different genes detectable on each chip. This should significantly raise the sensitivity and decrease the cost of such analyses.

170. Disclosed herein are the thermodynamic criteria for elimination of oligo-probes that are very likely poor RNA binders. The criteria are based on statistical analysis of hybridization of short 20 and 21 mer probes. Longer oligo-probes in the range from 50 to 150 mers can be also used for array experiments. Similar statistical analysis and thermodynamic filtration schemes can be applied to hybridization data produced with long oligo-probes. It can reveal optimal thermodynamic criteria for long oligo-probe design at different experimental conditions.

171. Target RNA secondary structure can also play an important role in selection of the most potent RNA binders. FIG. 4 demonstrates that many efficient RNA binders are lost during the steps of thermodynamic filtration performed in this study. It is likely that taking into consideration thermodynamic properties related to RNA secondary structure can diminish this loss. However, the analysis performed in this study reveals that oligo-probes with a high probability of being efficient RNA binders in array experiments can still be selected without consideration of the thermodynamic properties related to RNA secondary structure.

172. Thermodynamic filtration can dramatically increase the proportion of oligonucleotides with efficient RNA binding. As illustrated in FIGS. 4 and 6 and in Example 1, the proportions of efficient binders among the oligonucleotides in both experimental datasets are small (approximately 14% for dataset 1 and 10% for dataset 2). However, these proportions can be increased up to 70%, or even more, if a set of oligonucleotides that form stable duplexes with RNA and little self-structure are selected.

173. Removing subsets of oligonucleotides with low probability of hybridizing efficiently with their RNA target is important but is not the only problem relevant to probe design algorithms. Another important issue is elimination of the oligonucleotides that can cross hybridize with other genes. Modern algorithms include a BLAST search for dealing with the problem. The limitations of BLAST or similar programs are due to the absence of well-defined criteria for the prediction of hybridization. For optimal solution of this problem, an efficient thermodynamic predictor of hybridization intensity is needed.

174. Statistical analysis was performed to find out what range of values of dG°_{T} of DNA-RNA duplex stability of oligo-probes with little self-structure is optimal for this purpose. Two subsets from dataset 3 were created. Both subsets include only oligo-probes with little self-structure (dG°₂₅≧8 kcal/mol for inter-molecular structures and dG°₂₅≧11 kcal/mol for intra-molecular structures). The first subset includes oligo-probes with dG°₂₅ values of DNA-RNA duplex stability ranging from 0 to −10 kcal/mol. The second subset includes oligo-probes with dG°₂₅ values of DNA-RNA duplex stability ranging from −10 to 40 kcal/mol. The correlation between the values of hybridization intensities of the oligo-probes and the values of dG°₂₅ of DNA-RNA duplex stability was absent in the first subset and was highly significant in the second with a correlation coefficient of 0.7. The scatter plot with correlation trend-line for subset 2 from dataset 3 is presented in FIG. 7.

175. Statistical analysis reveals that the calculated value of dG°₂₅ of DNA-RNA duplex stability in the range from −10 to −40 kcal/mol can be considered as a predictor of oligo-probe hybridization intensity for the molecules with minimum self-structure. So the intensity of cross hybridization between these oligo-probes and partially complementary target sequences can be predicted after calculation of thermodynamic values. The scheme for this prediction is shown in FIG. 8. This scheme should be helpful for the discrimination of oligo-probes into candidates with strong or weak cross-hybridization potentials. The application of this scheme is limited to the conditions in which dataset 3 was obtained.

176. In conclusion, statistical analysis of large sets of hybridization data suggests that thermodynamic evaluation of oligonucleotide properties can be used to avoid poor RNA binders. This analysis also indicates that thermodynamic evaluation of oligonucleotide properties can be directly linked to the solution of the cross-hybridization problem. So thermodynamic calculations can be helpful for optimization of hybridization sensitivity and specificity of the oligo-probes. However, much more experimental data and software optimization are needed before cross-hybridization potentials of the oligo-probes can be reliably calculated for the range of hybridization conditions.

### 2. Example 2 Thermodynamic Criteria for High Hit Rate Antisense Oligonucleotide Design

a) Materials and Methods
   (1) Databases

177. For this work, two databases were used. The first one includes data from antisense oligonucleotide screening experiments reported in the literature (Giddings, M. C., et al., (2000), Bioinformatics, 16, 843-844). This database is available on the Web (http://antisense.genetics.utah.edu/). The second database utilizes the data from experiments performed at Isis Pharmaceuticals and were not yet reported in the literature. These databases include activity values and antisense oligonucleotide sequences. Activity value is expressed as the ratio of the level of a particular MRNA or protein measured in cells after treatment with the experimental antisense oligonucleotide versus the level of the same mRNA or protein measured in untreated cells. There are 316 oligonucleotides in the first database and 908 in the second.
(2) Thermodynamic calculations

178. Thermodynamic properties for oligonucleotides and relevant duplexes were calculated using the programs OligoWalk (Mathews, D. H., et al., (1999), RNA, 5, 1458-1469) and OligoScreen from the package RNAstructure 3.5 (http://128.151.176.70/RNAstructure.html). OligoWalk predicts the equilibrium affinity of complementary DNA or RNA oligonucleotides to an RNA target by calculating dG°ₒᵥₑᵣₐₗₗ values. These dG°ₒᵥₑᵣₐₗₗ values are calculated by consideration of dG°₃₇ values relevant to the predicted stability of the oligonucleotide-target duplex and the competition with predicted secondary structure of both the target and the oligonucleotide. Both dG°₃₇ values relevant to inter- and intra-molecular oligonucleotide self-structures are considered at a user-defined concentration. One thousand suboptimal structures were created for each mRNA target molecule. The disruption in RNA secondary structures included the free energy required for target rearrangement. OligoScreen (http://rna.chem.rochester.edu/) considers only the predicted stability of the oligonucleotide-target duplex and the competition with predicted secondary structure of the oligonucleotide without consideration of target RNA secondary structure. For determination of dG°₃₇, both programs use thermodynamic parameters for the nearest-neighbor model (Xia, T., et al., (1998), Biochemistry, 37, 14719-14735; SantaLucia, J., Jr (1998), Proc. Natl Acad. Sci. USA, 95, 1460-1465; SantaLucia, J., Jr, et al., (1996), Biochemistry, 35, 3555-3562; Allawi, H. T. and SantaLucia, J., Jr (1997), Biochemistry, 36, 10581-10594; Sugimoto, N., et al., (1995), Biochemistry, 34, 11211-11216; Luebke, K. J., et al., (2003), Nucleic Acids Res., 31, 750-758).
(3) Statistical analysis

179. Statistical tools from Excel (Microsoft, Inc.) were used for correlation analysis (t-test) and scatter plot data presentations.
b) Results

180. Statistical analysis has been performed on data collected from more than 1000 experiments with phosphorothioate-modified antisense oligonucleotides. Oligonucleotides that form stable duplexes with RNA [free energies (dG°₃₇)≦30 kcal/mol] and have small self-interaction potential are statistically more likely to be active than molecules that form less stable oligonucleotide-RNA hybrids or more stable self-structures. To achieve optimal statistical preference, the values for self-interaction should be (dG°₃₇)≧8 kcal/mol for inter-oligonucleotide pairing and (dG°₃₇)≧1.1 kcal/mol for intra-molecular pairing. Selection of oligonucleotides with these thermodynamic values in the analyzed experiments would have increased the proportion of active oligonucleotides by as much as 6-fold.

181. The equilibrium affinity of an oligonucleotide for target RNA is influenced by the stability of the potential RNA-DNA duplex and by the stability of competing structures including the oligonucleotide self-structure and the target RNA structure. The program OligoWalk (Mathews, D. H., et al., (1999), RNA, 5, 1458-1469) calculates dG°₃₇ values for each of these structures. In addition, dG°ₒᵥₑᵣₐₗₗ, the overall Gibbs free energy change of RNA binding at 37° C. for each oligonucleotide, is determined. These dG°ₒᵥₑᵣₐₗₗ values are calculated by consideration of dG°₃₇ values relevant to the predicted stability of the oligonucleotide-target duplex and the competition with predicted secondary structure of both the target and the oligonucleotide. Both dG°₃₇ values relevant to inter- and intra-molecular oligonucleotide self-structures are considered at a user-defined concentration. The efficiency of oligonucleotide-RNA binding correlated positively with the stability of the potential RNA-DNA duplex and correlated negatively with the stabilities of the oligonucleotide and mRNA secondary structures. Thus dG°ₒᵥₑᵣₐₗₗ correlated with experimental efficacy of the oligonucleotides better than any individual parameter.

182. The findings for the database of experiments reported in the literature are shown in Table 3. Surprisingly, the correlation between values of dGₒᵥₑᵣₐₗₗ and antisense oligonucleotide efficacy is very weak. Moreover, the stability of RNA secondary structures that must be disrupted for oligonucleotide-RNA helix formation does not correlate significantly with antisense efficacy. However, significant correlation was detected between antisense efficacy and dG°_{T} values associated with the stability of oligonucleotide self-structures and oligonucleotide-RNA duplexes.

TABLE 3
Correlations between thermodynamic properties of oligonucleotides and
their antisense activity

| | Correlation coefficient | Significance |
|---|---|---|
| ΔG°₃₇ overall versus ln(activity) | 0.17 | 0.01 |
| ΔG°₃₇ duplex versus ln(activity) | 0.24 | 2.3 × 10⁻⁵ |
| ΔG°₃₇ oligo intra-molecular | −0.12 | 0.03 |
| structure versus ln(activity) | | |
| ΔG°₃₇ oligo inter-molecular | −0.16 | 0.005 |
| structure versus ln(activity) | | |
| ΔG°37 target RNA secondary | No significant correlation | |
| structure versus ln(activity) | | |

184.

185.

186. The lack of correlation between efficacy and the stability of mRNA secondary structure may be due to inaccuracies in the mRNA secondary structure prediction and other factors discussed previously (Mathews, D. H., et al., (1999), RNA, 5, 1458-1469). Because no correlation was found for the predicted RNA secondary structure stability with antisense activity, and because the theoretical prediction of RNA secondary structure by free energy minimization is the most time consuming step of the calculations, further statistical analysis focused on thermodynamic parameters of the oligonucleotides and their duplexes with the target RNA. The previous studies of hybridization data produced with oligo-probes immobilized on arrays demonstrated that consideration of duplex stability between DNA and RNA, as well as considerations of oligonucleotide self-structure stability, can be sufficient for elimination of oligo-probes that hybridize poorly with the targets (Luebke, K. J., et al., (2003), Nucleic Acids Res., 31, 750-758).

187. Scatter plots (FIG. 9) illustrate the relationship between activity and thermodynamic properties of antisense oligonucleotides from both the published and Isis databases. Since the slope of the trend line in scatter plots indicates the existence of a correlation between two variables, a correlation between thermodynamic evaluation of oligonucleotide-RNA duplex stability and antisense efficacy is evident for both databases (FIG. 9, top two plots), especially for subsets of data in the range of dG°_{37 duplex} values from −30 to −10 kcal/mol. Flattening trend lines for subsets of data with dG°_{37 duplex} values<−30 kcal/mol indicate a very weak correlation, or its absence. Categorization of databases into two groups was done with dG°_{37 duplex}=−30 kcal/mol as a cut off point. The first group included oligonucleotides that target RNA with less favorable free energy for duplex formation (dG°_{37 duplex} values ranging from −30 to −10 kcal/mol), i.e. oligonucleotides that form less stable duplexes with RNA. The second group includes oligonucleotides that target RNA with more favorable free energy for duplex formation (dG°_{37 duplex} ranging from −40 to −30 kcal/mol), i.e. oligonucleotides that form more stable duplexes with RNA. The second group in each database is smaller than the first group (30 and 16% from the total number of molecules in the published and Isis data, respectively). For both databases, positive correlations between oligonucleotide activity and absolute values of dG°_{37 duplex} for oligonucleotide-RNA duplexes were significant for the first group and not significant for the second (Table 4). In contrast, negative correlations between oligonucleotide activities and absolute dG°₃₇ values of oligonucleotide self-pairing were undetectable in the first group, but were highly significant for the second (Table 4). The relevant scatter plots (FIG. 9, middle and bottom plots) demonstrate the relationship of activity of antisense oligonucleotides and thermodynamic evaluations of their self-pairing potentials. The slopes of the trend lines indicate the existence of a negative correlation between these variables for the second group of molecules. As mentioned earlier, relevant correlations were not detected for oligonucleotides from group 1, and the scatter plots with flat trend lines are not shown.

TABLE 4
Correlations between thermodynamic properties of antisense
oligonucleotides and their antisense activities for two experimental databases

| | | | |
|---|---|---|---|
| | | Number of | |
| Correlation | | oligos in the | (G + C)/ |
| coefficient | Significance | group | (A + G + C + T) |

Group 1 oligos that are forming less stable duplexes with target RNA (ΔG°₃₇ >−30 kcal/mol)

| | | | | | |
|---|---|---|---|---|---|
| Published | ΔG°₃₇ of oligo-target | 0.36 | 0.00017 | 219 | 50 |
| data | duplex versus | | | | |
| | ln(activity) | | | | |
| | ΔG°₃₇ of oligo intra- | Significant | | | |
| | molecular structure | correlation is | | | |
| | versus ln(activity) | absent | | | |
| | ΔG°₃₇ of oligo intra- | Significant | | | |
| | molecular structure | correlation is | | | |
| | versus ln(activity) | absent | | | |
| Isis data | ΔG°₃₇ of oligo target | 0.35 | 2 × 10⁻²³ | 762 | 44 |
| | duplex versus | | | | |
| | ln(activity) | | | | |
| | ΔG°₃₇ of oligo intra- | Significant | | | |
| | molecular structure | correlation is | | | |
| | versus ln(activity) | absent | | | |
| | ΔG°₃₇ of oligo-intra- | Significant | | | |
| | molecular structure | correlation is | | | |
| | versus ln(activity) | absent | | | |

Group 2 oligos that are forming more stable duplexes with target RNA (ΔG°₃₇ ≦−30 kcal/mol)

| | | | | | |
|---|---|---|---|---|---|
| Published | ΔG°₃₇ of oligo target | Significant | | 97 | 68 |
| data | duplex versus | correlation is | | | |
| | ln(activity) | absent | | | |
| | ΔG°₃₇ of oligo intra- | 0.37 | 0.00017 | | |
| | molecular structure | | | | |
| | versus ln(activity) | | | | |
| | ΔG°₃₇ of oligo intra- | −0.27 | 0.006 | | |
| | molecular structure | | | | |
| | versus ln(activity) | | | | |
| Isis data | ΔG°₃₇ of oligo target | Significant | | 146 | 73 |
| | duplex versus | correlation is | | | |
| | ln(activity) | absent | | | |
| | ΔG°₃₇ of oligo intra- | −0.22 | 0.007 | | |
| | molecular structure | | | | |
| | versus ln(activity) | | | | |
| | ΔG°₃₇ of oligo intra- | −0.3 | 0.003 | | |
| | molecular structure | | | | |
| | versus ln(activity) | | | | |

189. The list of potential explanations for the scatter in groups 1 and 2 in FIG. 9 include: variations in local secondary structure stabilities of RNA targets that were not picked up by OligoWalk, variations in uptake of oligonucleotides in different experiments, differential degradation in cells, or variations in intensities of non-specific interactions with undesired RNA targets.

190. The results of the correlation analysis for the oligonucleotides in the database of published data are presented graphically in FIG. 10, and the results for the database of Isis unpublished data are in FIG. 11. For both databases, the proportion of oligonucleotides with high antisense efficacy is larger in the group predicted to form more stable oligonucleotide-RNA duplexes than in the group that forms less stable hybrids. FIGS. 10 and 11 also graphically illustrate a negative correlation between antisense activity and the propensity for formation of self-structure by the group of oligonucleotides that are also able to form stable oligo-RNA duplexes. The thermodynamic parameters for phosphorothioate-modified DNA oligonucleotide hybridization are not available from the literature, and thus the parameters for non-modified DNA were used as an approximation. It is possible that a specific set of parameters for phosphorothioates would improve the correlation with antisense activity.

191. Oligonucleotide self-structure formation can compete with oligonucleotide binding to target RNA. During antisense oligonucleotide experiments, the concentrations of oligonucleotides are usually much higher than those of the relevant mRNAs. Therefore, oligonucleotide self-interaction may decrease the ‘hit rate’. Among the oligos that form the more stable duplexes with RNA, those which are predicted to form strong intra- and inter-molecular self-structures are not as active as those with little self-structure.

192. Another issue is why self-structure is a problem for the second group of oligonucleotides that can form more stable duplexes with RNA, but not a problem for oligonucleotides from the first group that can form less stable duplexes with the target. The reason is probably that oligonucleotides from the second group are more frequently G +C-rich molecules (Table 4) and thus are more likely to adopt stable self-structures. In contrast, oligonucleotides from the first group that form the less stable duplexes with target RNA are less frequently G +C-rich, so the proportion of those with stable self-structures is rather small. As a result of this difference in composition, the proportion of oligonucleotides with stable self-structure is also much higher among those that form stable duplexes with RNA. A large proportion of highly structured oligonucleotides in the second group of molecules is related to strong, and statistically detectable, negative effects on antisense hit rate. Correspondingly, a small proportion of structured oligonucleotides in the first group of molecules is related to undetectable negative effects on the hit rate.

193. Thermodynamic evaluations of both oligonucleotide intra- and inter-molecular self-interacting properties are strongly correlated with each other. Steep trend line slopes of scatter plots (FIG. 12), and highly significant correlation coefficients of 0.65 and 0.5, demonstrate this for both databases. Usually, if two variables are highly correlated, only one is sufficient for predictive purposes. However, with antisense oligonucleotides, it was found that both thermodynamic criteria for self-structure-forming potentials are simultaneously useful for efficient discrimination into categories that mainly contain either the most active molecules, or categories that contain the non-active ones. The statistical results presented indicate that using values for the predicted stability of duplexes of oligonucleotides with their target RNA, and corresponding values for oligonucleotide self-structure, can dramatically increase the proportion of active antisense oligos in trial and error screening experiments. If oligonucleotides in the optimal range described above had been used, the ‘hit rate’ would have been three times higher for the published data set and six times higher for the unpublished data from Isis Pharmaceuticals (FIG. 13).

### 3. Example 3 Identification of Conserved Regions in Multiple Sequences Alignments Thermodynamically Suitable for Targeting by Oligonucleotides: Initial Application to HIV Gag RNA

a) Materials and methods
   (1) Consensus sequence and multiple sequence alignments 194. Consensus sequence s for HIV-1 variants (group M) and multiple sequence alignments (Gaschen, B., et al., (2002) Bioinformatics, 17, 415-418) that were created by Los Alamos Laboratory staff were used in this work: These sequences can be found at http://hiv-web.1an1.gov/content/hiv-db/CONSENSUS/M_GROUP/Consensus.html and http://hiv-web.1an1.gov/content/hiv-db/ALIGN_CURRENT/ALIGN-INDEX.html. All of these sequences located at this site are herein incorporated by reference in their entireties.
   (2) Plot of conservation

195. The average percentage of conservation of each consecutive 30 nucleotides in multiple sequence alignments (based on division of the sum of percentage conservation of each nucleotide by the number of nucleotides) was calculated using the program created for this study.
(3) Evaluation of the potential for intra-molecular and inter-molecular self-interaction of DNA oligonucleotides.

Calculations of thermodynamic properties of oligonucleotides were done with the help of OligoWalk program from RNAStucture 3.7 program package (Mathews, D. H., et al., (1999) RNA, 5, 1458-1469) http://128.151.176.70/RNAstructure.html.
(4) Evaluation of pairing potentials among DNA oligonucleotides and target RNA variants 196. A computer program AlignScan was created to evaluate, the ΔG°₃₇ calculations, the pairing potential of each DNA consensus fragment with all divergent RNA variants. The program requires aligned sequence variants as an input file. It also requires fragment sequence lengths as an input parameter. ΔG°₃₇ values are calculated for all complementary duplexes between each successive fragment of consensus sequence and the corresponding fragment in all sequence variants. AlignScan output displays all consensus oligonucleotides of given length from the consensus sequence with accompanying ΔG°₃₇ values for duplexes between each oligonucleotide and the corresponding complementary target variants. The difference between the ΔG°₃₇ value of the consensus duplex and ΔG°₃₇ value of least favorable duplex for the target RNA variants within M group is also displayed.

197. The program was applied to the HIV-1 gag gene where it was used as part of a thermodynamic analysis to discriminate between conserved regions for their potential as target sequences for hybrid formation. The output files can be further processed with Excel (Microsoft, USA).
b) Results

198. The scheme developed for discrimination of conserved regions in multiple RNA sequence variants RNA target fragments is based on their potential to serve as efficient hybridization targets for oligonucleotides. It involves several steps and employs sequential filtering procedures. First, creation of a consensus sequence of RNA or DNA from aligned sequence variants with specification of the lengths of fragments to be used as oligonucleotides in the analyses. Second, selection of fragments in consensus sequence with homology, for the aligned multiple RNA sequence variants, greater than a defined threshold. Third, selection of DNA oligonucleotides that have pairing potential, greater than a defined threshold, with all variants of the aligned RNA sequences. Fourth, elimination of DNA oligonucleotides that have self-pairing potentials for intra- and inter-molecular interactions greater than defined thresholds. The consensus RNA sub-sequences complementary to the remaining set of oligonucleotides are preferred potential targets for hybridization.

199. The discrimination scheme described above was applied to the HIV-1 gag genes where the need to identify hybridization targets is obvious. For the first set of results the fragment length was arbitrarily chosen to be 30 nts. For each successive fragment of consensus sequence, the average conservation values were calculated (as described in Methods) and plotted as a histogram (FIG. 14B). This histogram demonstrates that the conservation values for 30 nucleotide gag windows vary from 68% to 95%. Approximately one half of 30-mers from the consensus gag sequence have values of conservation higher than 87%. This set of most conserved regions was used for the next steps of thermodynamic discrimination analysis. The oligonucleotides that form stable duplexes with RNA (free energies (ΔG°₃₇)≧30 kcal/mol) and little self structure with (ΔG°₃₇)≦−8 kcal/mol for inter- oligonucleotide pairing and (AG°₃₇)≧−1.1 kcal/mol for intramolecular pairing were selected.

200. Theoretically optimal hybridization targets are shown in FIG. 14. The last nucleotide of each fragment is highlighted in the consensus sequence (A) or conservation histogram (B). Only sub-set of conserved target fragments in gag gene is “optimal” for hybridization with oligonucleotides. FIG. 14B shows that only some of the spikes in the histogram that corresponds to most conserved regions in gag are highlighted.

201. It is interesting that the length of oligonucleotides correlated with the numbers of theoretically optimal RNA targets obtained after conservation and thermodynamic selection procedures. More optimal targets can be detected for longer oligonucleotides (FIG. 15).

202. The consensus sequence of gag yields total number of 23704 complementary oligonucleotides ranging in size from 20 to 35 mers. The set of 1747 oligonucleotides that is 14 times smaller than initial one remains after steps of homology and thermodynamic discrimination described here. The target regions for the oligonucleotides from this set are visualized in FIG. 14 with the last nucleotide of each fragment being highlighted.

203. At 37° C. the proportion of good binders among the oligonucleotides in experimental database is small (approximately 14%), however this proportions can be increased up to 70% or even more if the set of oligonucleotides that form stable RNA duplexes and little self-structure had been selected.

204. The temperature used for the experiments from which the thermodynamic thresholds were derived, is 37° C. Application of these thresholds in the current work yields hybridization target regions that are optimal for the same temperature. The list of selected regions for oligonucleotide hybridization targeting is relevant to procedures that involve oligonucleotide RNA pairing at about 37° C. such as branch DNA detection technology and often reverse transcription. For PCR that requires higher temperature, other thermodynamic thresholds can be used. (Additional thermodynamic discrimination steps should be performed for elimination sets of forward and reverse primers that can interact with each other.)

205. Chemically synthesized consensus oligonucleotides for targets that were selected after rounds of discrimination analysis, can be immobilized on an array and subjected to hybridizations with labeled RNA of different representatives of the HIV-1 M group. These hybridizations should reveal oligonucleotides with consistent high affinity toward different RNA variants. These molecules should be prime candidates for sensitive viral detection procedures or experiments that require efficient oligonucleotide-RNA interaction for the broad range of viral variants. The set of oligonucleotides for gag that remains after homology and thermodynamic selection is 14 times smaller than the initial set of all possible oligonucleotides in this range. Around 70% of the oligonucleotides from this theoretically selected set will demonstrate consistency in hybridization behavior with different representatives of group M viruses.

H. REFERENCES

1. Walker, G. T., Fraiser, M. S., Schram, J. L., Little, M. C., Nadeau, J. G. and Malinowski, D. P. (1992) Strand displacement amplification--an isothermal, in vitro DNA amplification technique Nucleic Acids Res, 20, 1691-1696.

2. Walker, G. T., Little, M. C., Nadeau, J. G. and Shank, D. D. (1992) Isothermal in vitro amplification of DNA by a restriction enzyme/DNA polymerase system Proc Natl Acad Sci USA, 89, 392-396.

3. Kacian, D. L. and Fultz, T. J.(1995) Nucleic Acid Sequence Amplification Methods U.S. Pat. No. 5,399,491.

4. Compton, J. (1991) Nucleic acid sequence-based amplification Nature, 350, 91-92.

5. Arnold, L. J., Jr., Hammond, P. W., Wiese, W. A. and Nelson, N. C. (1989) Assay formats involving acridinium-ester-labeled DNA probes Clin Chem, 35, 1588-1594.

6. Urdea, M. S., Wilber, J. C., Yeghiazarian, T., Todd, J. A., Kern, D. G., Fong, S. J., Besemer, D., Hoo, B., Sheridan, P. J., Kokka, R. et al. (1993) Direct and quantitative detection of HIV-1 RNA in human plasma with a branched DNA signal amplification assay Aids, 7 Suppl 2, S11-14.

7. Urdea, M. S. (1994) Branched DNA signal amplification Biotechnology (N Y), 12, 926-928.

8. DeLong, E. F., Wickham, G. S. and Pace, N. R. (1989) Phylogenetic stains: ribosomal RNA-based probes for the identification of single cells Science, 243, 1360-1363.

9. Amann, R. I., Ludwig, W. and Schleifer, K. H. (1995) Phylogenetic identification and in situ detection of individual microbial cells without cultivation Microbiol Rev, 59, 143-169.

10. Chew, C. B., Zheng, F., Byth, K., Van Asten, M., Workman, C. and Dwyer, D. E. (1999) Comparison of three commercial assays for the quantification of plasma HIV-1 RNA from individuals with low viral loads [letter] Aids, 13, 1977-1978.

11. Debyser, Z., Van Wijngaerden, E., Van Laethem, K., Beuselinck, K., Reynders, M., De Clercq, E., Desmyter, J. and Vandarnme, A. M. (1998) Failure to quantify viral load with two of the three commercial methods in a pregnant woman harboring an HIV type 1 subtype G strain AIDS Res Hum Retroviruses, 14, 453-459.

12. Higgins, D. G. and Sharp, P. M. (1988) CLUSTAL: a package for performing multiple sequence alignment on a microcomputer Gene, 73, 237-244.

13. Gaschen, B., Kuiken, C., Korber, B. and Foley, B. (2002) Bioinformatics, 17,415-418.

14. Mathews, D. H., Burkard, M. E., Freier, S. M., Wyatt, J. R. and Turner, D. H.(1999) Predicting oligonucleotide affinity to nucleic acid targets. RNA, 5, 1458-1469.

15. Lucas, K., Busch, M., Mossinger, S. and Thompson, J. A. (1991) An improved microcomputer program for finding gene- or gene family- specific oligonucleotides suitable as primers for polymerase chain reactions or as probes Comput Appl Biosci, 7, 525-529.

16. Dopazo, J., Rodriguez, A., Saiz, J. C. and Sobrino, F. (1993) Design of primers for PCR amplification of highly variable genomes Comput Appl Biosci, 9, 123-125.

17. Proutski, V. and Holmes, E. C. (1996) Primer Master: a new program for the design and analysis of PCR primers Comput Appl Biosci, 12, 253-255.

18. Kel, A., Ptitsyn, A., Babenko, V., Meier-Ewert, S. and Lehrach, H. (1998) A genetic algorithm for designing gene family-specific oligonucleotide sets used for hybridization: the G protein-coupled receptor protein superfamily Bioinformatics, 14, 259-270.

19. Gibbs, A., Armstrong, J., Mackenzie, A. M. and Weiller, G. F. (1998) The GPRIME package: computer programs for identifying the best regions of aligned genes to target in nucleic acid hybridisation-based diagnostic tests, and their use with plant viruses J Virol Methods, 74, 67-76.

1. Williams, J. C., Case-Green, S. C., Mir, K. U. and Southern, E. M. (1994) Studies of oligonucleotide interactions by hybridization to arrays: the influence of dangling ends on duplex yield. Nucleic Acids Res., 22, 1365-1367.[Abstract]

2. Southern, E. M., Case-Green, S. C., Elder, J. K., Johnson, M., Mir, K. U., Wang, L. and Williams, J. C. (1994) Arrays of complementary oligonucleotides for analyzing the hybridization behavior of nucleic acids. Nucleic Acids Res., 22, 1368-1373.[Abstract]

3. Southern, E. M. (2002) History and overview. Methods Mol. Biol., 170, 1-15.[Medline]

4. Sohail, M., Akhtar, S. and Southern, E. M. (1999) The folding of large RNAs studied by hybridization to arrays of complementary oligonucleotides. RNA, 5, 646-655.[Abstract/Free Full Text]

5. Sohail, M. and Southern, E. M. (2002) Using oligonucleotide scanning arrays to find effective antisense reagents. Methods Mol. Biol., 170, 181-199.[Medline]

6. Sohail, M., Hochegger, H., Klotzbucher, A., Guellec, R. L., Hunt, T. and Southern, E. M. (2002) Antisense oligonucleotides selected by hybridization to scanning arrays are effective reagents in vivo. Nucleic Acids Res., 29, 2041-2051.[Abstract/Free Full Text]

7. Southern, E., Mir, K. and Shchepinov, M. (1999) Molecular interactions on microarrays. Nature Genet., 21, 5-9.[CrossRef][Medline]

8. Mathews, D. H., Burkard, M. E., Freier, S. M., Wyatt, J. R. and Turner, D. H. (1999) Predicting oligonucleotide affinity to nucleic acid targets. RNA, 5, 1458-1469.[Abstract/Free Full Text]

9. SantaLucia, J., Jr, Allawi, H. T. and Seneviratne, P. A. (1996) Improved nearest-neighbor parameters for predicting DNA duplex stability. Biochemistry, 35, 3555-3562.[CrossRef][Medline]

10. Allawi, H. T. and SantaLucia, J., Jr (1997) Thermodynamics and NMR of internal G.T mismatches in DNA. Biochemistry, 36, 10581-10594.[CrossRef][Medline]

11. Allawi, H. T. and SantaLucia, J., Jr (1998) Thermodynamics of internal C.T mismatches in DNA. Nucleic Acids Res., 26, 2694-2701.[Abstract/Free Full Text]

12. Allawi, H. T. and SantaLucia, J., Jr (1998) Nearest neighbor thermodynamic parameters for internal G.A mismatches in DNA. Biochemistry, 37, 2170-2179.[CrossRef][Medline]

13. Allawi, H. T. and SantaLucia, J., Jr (1998) Nearest-neighbor thermodynamics of internal A.C mismatches in DNA: sequence dependence and pH effects. Biochemistry, 37, 9435-9444. [CrossRef][Medline]

14. Peyret, N., Seneviratne, P. A., Allawi, H. T. and SantaLucia, J., Jr (1999) Nearest-neighbor thermodynamics and NMR of DNA sequences with internal A.A, C.C, G.G, and T.T mismatches. Biochemistry, 38, 3468-3477.[CrossRef][Medline]

15. SantaLucia, J., Jr (1998) A unified view of polymer, dumbbell, and oligonucleotide DNA nearest-neighbor thermodynamics. Proc. Natl Acad. Sci. USA, 95, 1460-1465.[Abstract/Free Full Text]

16. Sugimoto, N., Nakano, S., Katoh, M., Matsumura, A., Nakamuta, H., Ohmichi, T., Yoneyama, M. and Sasaki, M. (1995) Thermodynamic parameters to predict stability of RNA/DNA hybrid duplexes. Biochemistry, 34, 11211-11216.[Medline]

17. Shannon, K. and Wolber, P. (2002) Method for evaluating oligonucleotide probe sequences. U.S. Pat. No. 6,251,588.

18. Walton, S. P., Stephanopoulos, G. N., Yarmush, M. L. and Roth, C. M. (1999) Prediction of antisense oligonucleotide binding affinity to a structured RNA target. Biotechnol. Bioeng., 65, 1-9. [CrossRef][Medline]

19. Jayaraman, A., Walton, S. P., Yarmush, M. L. and Roth, C. M. (2002) Rational selection and quantitative evaluation of antisense oligonucleotides. Biochim. Biophys. Acta, 1520, 105-114.[Medline]

20. Walton, S. P., Stephanopoulos, G. N., Yarmush, M. L. and Roth, C. M. (2002) Thermodynamic and kinetic characterization of antisense oligodeoxynucleotide binding to a structured mRNA. Biophys. J., 82, 366-377.[Abstract/Free Full Text]

21. Luebke, K. J., Balog, R. P. and Garner, H. R. (2003) Prioritized selection of oligodeoxyribonucleotide probes for efficient hybridization to RNA transcripts. Nucleic Acids Res., 31, 750-758.[Abstract/Free Full Text]

1.

Sczakiel, G. and Tabler, M. (1997) Computer-aided calculation of the local folding potential of target RNA and its use for ribozyme design. Methods Mol. Biol., 74, 11-15.[Medline]

2. Patzel, V., Steidl, U., Kronenwett, R., Haas, R. and Sczakiel, G. (1999) A theoretical approach to select effective antisense oligodeoxyribonucleotides at high statistical probability. Nucleic Acids Res., 27, 4328-4334.[Abstract/Free Full Text]

3. Lehmann, M. J., Patzel, V. and Sczakiel, G. (2000) Theoretical design of antisense genes with statistically increased efficacy. Nucleic Acids Res., 28, 2597-2604.[Abstract/Free Full Text]

4. Scherr, M., Rossi, J. J., Sczakiel, G. and Patzel, V. (2000) RNA accessibility prediction: a theoretical approach is consistent with experimental studies in cell extracts. Nucleic Acids Res., 28, 2455-2461. [Abstract/Free Full Text]

5. Sczakiel, G. (2000) Theoretical and experimental approaches to design effective antisense oligonucleotides. Front. Biosci., 5, 194-201.

6. Ding, Y. and Lawrence, C. E. (2002) Statistical prediction of single-stranded regions in RNA secondary structure and application to predicting effective antisense target sites and beyond. Nucleic Acids Res., 29, 1034-1046.[Abstract/Free Full Text]

7. Mathews, D. H., Burkard, M. E., Freier, S. M., Wyatt, J. R. and Turner, D. H. (1999) Predicting oligonucleotide affinity to nucleic acid targets. RNA, 5, 1458-1469.[Abstract/Free Full Text]

8. Giddings, M. C., Matveeva, O. V., Atkins, J. F. and Gesteland, R. F. (2000) ODNBase—a web database for antisense oligonucleotide effectiveness studies. Oligodeoxynucleotides. Bioinformatics, 16, 843-844. [Abstract]

9. Xia, T., SantaLucia, J., Jr, Burkard, M. E., Kierzek, R., Schroeder, S. J., Jiao, X., Cox, C. and Tumer, D. H. (1998) Thermodynamic parameters for an expanded nearest-neighbor model for formation of RNA duplexes with Watson-Crick base pairs. Biochemistry, 37, 14719-14735.[CrossRef][Medline]

10. SantaLucia, J., Jr (1998) A unified view of polymer, dumbbell and oligonucleotide DNA nearest-neighbor thermodynamics. Proc. Natl Acad. Sci. USA, 95, 1460-1465.[Abstract/Free Full Text]

11. SantaLucia, J., Jr, Allawi, H. T. and Seneviratne, P. A. (1996) Improved nearest-neighbor parameters for predicting DNA duplex stability. Biochemistry, 35, 3555-3562.[CrossRef][Medline]

12. Allawi, H. T. and SantaLucia, J., Jr (1997) Thermodynamics and NMR of internal G—T mismatches in DNA. Biochemistry, 36, 10581-10594.[CrossRef][Medline]

13. Sugimoto, N., Nakano, S., Katoh, M., Matsumura, A., Nakamuta, H., Ohmichi, T., Yoneyama, M. and Sasaki, M. (1995) Thermodynamic parameters to predict stability of RNA/DNA hybrid duplexes. Biochemistry, 34, 11211-11216.[Medline]

14. Luebke, K. J., Balog, R. P. and Gamer, H. R. (2003) Prioritized selection of oligodeoxyribonucleotide probes for efficient hybridization to RNA transcripts. Nucleic Acids Res., 31, 750-758.[Abstract/Free Full Text]

15. Matveeva, O. V., Shabalina, S. A., Nemtsov, V. A., Tsodikov, A. D., Gesteland, R. F. and Atkins, J. F. (2003) Thermodynamic calculations and statistical correlations for oligo-probes design. Nucleic Acids Res., 31, 4211-4217.[Abstract/Free Full Text]

What is claimed is:

1. A method of identifying a set of oligonucleotides that will hybridize with a target nucleic acid comprising, a) determining the dG of oligo-target binding for each potential oligonucleotide that can bind to the target at a determined temperature, b) selecting the oligonucleotides that have a dG of≦30 kcal/mol forming an oligo-target set of oligonucleotides, c) determining the dG at the determined temperature for the intramolecular interactions for the oligonucleotides in the oligo-target set and the dG at the determined temperature for the oligo-oligo intermolecular interactions for the oligonucleotides in the oligo target set, and d) selecting those oligonucleotides from the oligo-target set that have a dG for intermolecular interactions of ≧−8 kcal/mol and have an intramolecular dG of≧−1 kcal/mol, forming a target set of oligonucleotides.

2. The method of claim 1 further comprising the step of determining the length of the each oligonucleotide within the set prior to step a).

3. The method of claim 2, wherein the oligonucleotide length is 20 bases.

4. The method of claim 2, wherein the oligonucleotide length is 21 bases.

5. The method of claim 2, wherein the oligonucleotide length is 30 bases.

6. The method of claim 1, wherein the target temperature is between 25 degrees C. to 45 degrees C.

7. The method of claim 1, wherein the target temperature is between 28 degrees C. to 42 degrees C.

8. The method of claim 1, wherein the target temperature is between 32 degrees C. to 38 degrees C.

9. The method of claim 1, wherein the target temperature is 37 degrees Celsius.

10. The method of claim 1, wherein the target temperature is 25 degrees Celsius.

11. The method of claim 10, wherein step b) comprises selecting the oligonucleotides that have a dG of≦−35 kcal/mol forming an oligo-target set of oligonucleotides.

12. The method of claim 1, wherein the target nucleic acid is a consensus sequence of a set of target nucleic acids.

13. The method of claim 12, wherein prior to the step a), the method further comprises the step of determining the consensus sequence of the target nucleic acid.

14. The method of claim 12, wherein the method further comprises the step of adjusting the oligo-target set generated in step a) such that only 99% of the oligonucleotides in the oligo-target set meet the dG requirements.

15. The method of claim 12, wherein the method further comprises the step of adjusting the oligo-target set generated in step a) such that only 95% of the oligonucleotides in the oligo-target set meet the dG requirements.

16. The method of claim 12, wherein the method further comprises the step of adjusting the oligo-target set generated in step a) such that only 90% of the oligonucleotides in the oligo-target set meet the dG requirements.

17. The method of claim 12, wherein the method further comprises the step of adjusting the oligo-target set generated in step a) such that only 80% of the oligonucleotides in the oligo-target set meet the dG requirements.

18. The method of claim 12, wherein the target nucleic acid is a viral nucleic acid.

19. The method of claim 18, wherein the target nucleic acid is RNA.

20. The method of claim 18, wherein the target nucleic acid is DNA.

21. The method of claim 18, wherein the viral nucleic acid is derived from Adenoviridae, Arenaviridae, Astroviridae, Baculoviridae, Barnaviridae, Betaherpesvirinae, Bimaviridae, Bromoviridae, Bunyaviridae, Caliciviridae, Chordopoxvirinae, Circoviridae, Comoviridae, Coronaviridae, Cystoviridae, Corticoviridae, Entomopoxvirinae, Filoviridae, Flaviviridae, Fuselloviridae, Geminiviridae, Hepadnaviridae, Herpesviridae, Gammaherpesvirinae, Inoviridae, Iridoviridae, Leviviridae, Lipothrixviridae, Microviridae, Myoviridae, Nodaviridae, Orthomyxoviridae, Papovaviridae, Paramyxoviridae, Paramyxovirinae, Partitiviridae, Parvoviridae, Phycodnaviridae, Picornaviridae, Plasmaviridae, Pneumovirinae, Podoviridae, Polydnaviridae, Potyviridae, Poxviridae, Reoviridae, Retroviridae, Rhabdoviridae, Sequiviridae, Siphoviridae, Tectiviridae, Tetraviridae, Togaviridae, Tombusviridae, Totiviridae, or vaiant or strain thereof.

22. The method of claim 18, wherein the viral nucleic acid is derived from Mastadenovirus, Human adenovirus 2, Aviadenovirus, African swine fever virus, arenavirus, Lymphocytic choriomeningitis virus, Ippy virus, Lassa virus, Arterivirus, Human astrovirus 1, Nucleopolyhedrovirus, Autographa californica nucleopolyhedrovirus, Granulovirus, Plodia interpunctella granulovirus, Badnavirus, Commelina yellow mottle virus, Rice tungro bacilliform, Barnavirus, Mushroom bacilliform virus, Aquabirnavirus, Infectious pancreatic necrosis virus, Avibirnavirus, Infectious bursal disease virus, Entomobirnavirus, Drosophila X virus, Alfamovirus, Alfalfa mosaic virus, Ilarvirus, Ilarvirus Subgroups 1-10, Tobacco streak virus, Bromovirus, Brome mosaic virus, Cucumovirus, Cucumber mosaic virus, Bhanja virus Group, Kaisodi virus, Mapputta virus, Okola virus, Resistencia virus, Upolu virus, Yogue virus, Bunyavirus, Anopheles A virus, Anopheles B virus, Bakau virus, Bunyamwera virus, Bwamba virus, C virus, California encephalitis virus, Capim virus, Gamboa virus, Guama virus, Koongol virus, Minatitlan virus, Nyando virus, Olifantsvlei virus, Patois virus, Simbu virus, Tete virus, Turlock virus, Hantavirus, Hantaan virus, Nairovirus, Crimean-Congo hemorrhagic fever virus, Dera Ghazi Khan virus, Hughes virus, Nairobi sheep disease virus, Qalyub virus, Sakhalin virus, Thiafora virus, Crimean-congo hemorrhagic fever virus, Phlebovirus, Sandfly fever virus, Bujaru complex, Candiru complex, Chilibre complex, Frijoles complex, Punta Toro complex, Rift Valley fever complex, Salehabad complex, Sandfly fever Sicilian virus, Uukuniemi virus, Uukuniemi virus, Tospovirus, Tomato spotted wilt virus, Calicivirus, Vesicular exanthema of swine virus, Capillovirus, Apple stem grooving virus, Carlavirus, Carnation latent virus, Caulimovirus, Cauliflower mosaic virus, Circovirus, Chicken anemia virus, Closterovirus, Beet yellows virus, Comovirus, Cowpea mosaic virus, Fabavirus, Broad bean wilt virus 1, Nepovirus, Tobacco ringspot virus, Coronavirus, Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, Rat coronavirus, Turkey coronavirus, Rabbit coronavirus, Torovirus, Berne virus, Breda virus, Corticovirus, Alteromonas phage PM2, Pseudomonas Phage phi6, Deltavirus, Hepatitis delta virus, Dianthovirus Carnation ringspot virus, Red clover necrotic mosaic virus, Sweet clover necrotic mosaic virus, Enamovirus, Pea enation mosaic virus, Filovirus, Marburg virus, Ebola virus Zaire, Flavivirus, Yellow fever virus, Tick-borne encephalitis virus, Rio Bravo Group, Japanese encephalitis, Tyuleniy Group, Ntaya Group, Uganda S Group, Dengue Group, Modoc Group, Pestivirus, Bovine diarrhea virus, Hepatitis C virus, Furovirus, Soil-borne wheat mosaic virus, Beet necrotic yellow vein virus, Fusellovirus, Sulfobolus virus 1, Subgroup I, II, and m geminivirus, Maize streak virus, Beet curly top virus, Bean golden mosaic virus, Orthohepadnavirus, Hepatitis B virus, Avihepadnavirus, Alphaherpesvirinae, Simplexvirus, Human herpesvirus 1, Varicellovirus, Human herpesvirus 3, Cytomegalovirus, Human herpesvirus 5, Muromegalovirus, Mouse cytomegalovirus 1, Roseolovirus, Human herpesvirus 6, Lymphocryptovirus, Human herpesvirus 4, Rhadinovirus, Ateline herpesvirus 2, Hordeivirus, Barley stripe mosaic virus, Hypoviridae, Hypovirus, Cryphonectria hypovirus 1-EP713, Idaeovirus, Raspberry bushy dwarf virus, Inovirus, Coliphage fd, Plectrovirus, Acholeplasma phage L51, Iridovirus, Chilo iridescent virus, Chloriridovirus, Mosquito iridescent virus, Ranavirus, Frog virus 3, Lymphocystivirus, Lymphocystis disease virus flounder isolate, Goldfish virus 1, Levivirus, Enterobacteria phage MS2, Allolevirus, Enterobacteria phage Qbeta, Lipothrixvirus, Thermoproteus virus 1, Luteovirus, Barley yellow dwarf virus, Machlomovirus, Maize chlorotic mottle virus, Marafivirus, Maize rayado fino virus, Microvirus, Coliphage phiX174, Spiromicrovirus, Spiroplasma phage 4, Bdellomicrovirus, Bdellovibrio phage MAC 1, Chlamydiamicrovirus, Chlamydia phage 1, T4-like phages, coliphage T4, Necrovirus, Tobacco necrosis virus, Nodavirus, Nodamura virus, Influenzavirus A, B and C, Thogoto virus, Polyomavirus, Murine polyomavirus, Papillomavirus, Rabbit (Shope) Papillomavirus, Paramyxovirus, Human parainfluenza virus 1, Morbillivirus, Measles virus, Rubulavirus, Mumps virus, Pneumovirus, Human respiratory syncytial virus, Partitivirus, Gaeumannomyces graminis virus 019/6-A, Chrysovirus, Penicillium chrysogenum virus, Alphacryptovirus, White clover cryptic viruses 1 and 2, Betacryptovirus, Parvovirinae, Parvovirus, Minute mice virus, Erythrovirus, B19 virus, Dependovirus, Adeno-associated virus 1, Densovirinae, Densovirus, Junonia coenia densovirus, Iteravirus, Bombyx mori virus, Contravirus, Aedes aegypti densovirus, Phycodnavirus, 1-Paramecium bursaria Chlorella NC64A virus group, Paramecium bursaria chlorella virus 1, 2-Paramecium bursaria Chlorella Pbi virus, 3-Hydra viridis Chlorella virus, Enterovirus, Human poliovirus 1, Rhinovirus Human rhinovirus 1A, Hepatovirus, Human hepatitis A virus, Cardiovirus, Encephalomyocarditis virus, Aphthovirus, Foot-and-mouth disease virus, Plasmavirus, Acholeplasma phage L2, Podovirus, Coliphage T7, Ichnovirus, Campoletis sonorensis virus, Bracovirus, Cotesia melanoscela virus, Potexvirus, Potato virus X, Potyvirus, Potato virus Y, Rymovirus, Ryegrass mosaic virus, Bymovirus, Barley yellow mosaic virus, Orthopoxvirus, Vaccinia virus, Parapoxvirus, Orf virus, Avipoxvirus, Fowlpox virus, Capripoxvirus, Sheep pox virus, Leporipoxvirus, Myxoma virus, Suipoxvirus, Swinepox virus, Molluscipoxvirus, Molluscum contagiosum virus, Yatapoxvirus, Yaba monkey tumor virus, Entomopoxviruses A, B, and C, Melolontha melolontha entomopoxvirus, Amsacta moorei entomopoxvirus, Chironomus luridus entomopoxvirus, Orthoreovirus, Mammalian orthoreoviruses, reovirus 3, Avian orthoreoviruses, Orbivirus, African horse sickness viruses 1, Bluetongue viruses 1, Changuinola virus, Corriparta virus, Epizootic hemarrhogic disease virus 1, Equine encephalosis virus, Eubenangee virus group, Lebombo virus, Orungo virus, Palyam virus, Umatilla virus, Wallal virus, Warrego virus, Kemerovo virus, Rotavirus, Groups A-F rotaviruses, Simian rotavirus SA11, Coltivirus, Colorado tick fever virus, Aquareovirus, Groups A-E aquareoviruses, Golden shiner virus, Cypovirus, Cypovirus types 1-12, Bombyx mori cypovirus 1, Fijivirus, Fijivirus groups 1-3, Fiji disease virus, Fijivirus groups 2-3, Phytoreovirus, Wound tumor virus, Oryzavirus, Rice ragged stunt, Mammalian type B retroviruses, Mouse mammary tumor virus, Mammalian type C retroviruses, Murine Leukemia Virus, Reptilian type C oncovirus, Viper retrovirus, Reticuloendotheliosis virus, Avian type C retroviruses, Avian leukosis virus, Type D Retroviruses, Mason-Pfizer monkey virus, BLV-HTLV retroviruses, Bovine leukemia virus, Lentivirus, Bovine lentivirus, Bovine immunodeficiency virus, Equine lentivirus, Equine infectious anemia virus, Feline lentivirus, Feline immunodeficiency virus, Canine immunodeficiency virus Ovine/caprine lentivirus, Caprine arthritis encephalitis virus, Visna/maedi virus, Primate lentivirus group, Human immunodeficiency virus 1, Human immunodeficiency virus 2, Human immunodeficiency virus 3, Simian immunodeficiency virus, Spumavirus, Human spuma virus, Vesiculovirus, Vesicular stomatitis Indiana virus, Lyssavirus, Rabies virus, Ephemerovirus, Bovine ephemeral fever virus, Cytorhabdovirus, Lettuce necrotic yellows virus, Nucleorhabdovirus, Potato yellow dwarf virus, Rhizidiovirus, Rhizidiomyces virus, Sequivirus, Parsnip yellow fleck virus, Waikavirus, Rice tungro spherical virus, Lambda-like phages, Coliphage lambda, Sobemovirus, Southern bean mosaic virus, Tectivirus, Enterobacteria phage PRD1, Tenuivirus, Rice stripe virus, Nudaurelia capensis beta-like viruses, Nudaurelia beta virus, Nudaurelia capensis omega-like viruses, Nudaurelia omega virus, Tobamovirus, Tobacco mosaic virus (vulgare strain; ssp. NC82 strain), Tobravirus, Tobacco rattle virus, Alphavirus, Sindbis virus, Rubivirus, Rubella virus, Tombusvirus, Tomato bushy stunt, virus, Carmovirus, Carnation mottle virus, Turnip crinkle virus, Totivirus, Saccharomyces cerevisiae virus, Giardiavirus, Giardia lamblia virus, Leishmaniavirus, Leishmania brasiliensis virus 1-1, Trichovirus, Apple chlorotic leaf spot virus, Tymovirus, Turnip yellow mosaic virus, Umbravirus, Carrot mottle virus, or variant or strain thereof.

23. The method of claim 12, wherein the viral nucleic acid is derived from Human immunodeficiency virus 1, Human immunodeficiency virus 2, or Human immunodeficiency virus 3.

24. The method of claim 12, wherein the target nucleic acid is a bacterial nucleic acid.

25. The method of claim 24, wherein the bacterial nucleic acid is derived from Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholerae, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof.

26. The method of claim 24, wherein the target nucleic acid is RNA.

27. The method of claim 24, wherein the target nucleic acid is DNA.

28. The method of claim 12, wherein the target nucleic acid is a fungal nucleic acid.

29. The method of claim 28, wherein the fungal nucleic acid is derived from Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternate, or strain or variant thereof.

30. The method of claim 28, wherein the target nucleic acid is RNA.

31. The method of claim 28, wherein the target nucleic acid is DNA.

32. The method of claim 12, wherein the target nucleic acid is a parasite nucleic acid.

33. The method of claim 32, wherein the parasite nucleic acid is derived from Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica, or any strain or variant thereof.

34. A composition comprising a primer, wherein the primer comprises the sequence of set forth in position 1042-1065 of SEQ ID NO:1.
